(19) United States
(12) Patent Application Publication
(10) Pub. No.: US 2023/0271015 A1
(43) Pub. Date: Aug. 31, 2023

(54) Automated Selection of Electrodes and Stimulation Parameters in a Deep Brain Stimulation System Using Sensed Neural Potentials

(71) Applicant: Boston Scientific Neuromodulation Corporation, Valencia, CA (US)

(72) Inventors: Mahsa Malekmohammadi, Sherman Oaks, CA (US); Stephen Carcieri, Los Angeles, CA (US); Lisa Moore, Glendale, CA (US); Raul Serrano Carmona, Venice, CA (US); G. Karl Steinke, Valencia, CA (US); Leon Juarez Paz, Berlin (DE)

(21) Appl. No.: 18/175,312

(22) Filed: Feb. 27, 2023

Related U.S. Application Data
(60) Provisional application No. 63/268,656, filed on Feb. 28, 2022.

(51) Int. Cl. A61N 1/36; A61N 1/372

(52) U.S. Cl. CPC A61N 1/36185; A61N 1/36139; A61N 1/37241; A61N 1/0534

(57) ABSTRACT

A method for optimizing stimulation for a patient having a stimulator device such as a Deep Brain Stimulation (DBS) device is disclosed. Test stimulation is provided at initial combinations of lead positions and values of a stimulation parameter such as amplitude, neural potentials are measured for each combination, and a neural response score is determined using the measured neural potentials. A next combination of position and a value of the stimulation parameter to test is determined using the neural response scores. This process repeats iteratively until a stopping criterium is met. The neural response scores and then used determine optimal therapeutic stimulation for the patient. Neural response measurements can also be used to exclude certain positions or stimulation values during subsequent optimization testing.

### CROSS REFERENCE TO RELATED APPLICATIONS

This is a non-provisional application of U.S. Provisional Patent Application Ser. No. 63/268,656, filed Feb. 28, 2022, which is incorporated by reference in its entirety, and to which priority is claimed.

### FIELD OF THE INVENTION

This application relates to Implantable Stimulator Devices (ISD), and more specifically to an algorithm for selecting electrodes and stimulation parameters in an ISD such as a Deep Brain Stimulation (DBS) device.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Deep Brain Stimulation (DBS) system, such as that disclosed in U.S. Patent Application Publication 2020/0001091, which is incorporated herein by reference. However, the present invention may find applicability with any implantable neurostimulator device system, including Spinal Cord Stimulation (SCS) systems, Vagus Nerve Stimulation (VNS) system, Sacral Nerve Stimulation (SNS) systems, Peripheral Nerve Stimulation (PNS) systems, and the like.

A DBS system typically includes an Implantable Pulse Generator (IPG) 10 shown in FIG. 1A. The IPG 10 includes a biocompatible device case 12 that holds the circuitry and a battery 14 for providing power for the IPG to function, although the IPG 10 can also lack a battery and can be wirelessly powered by an external source. The IPG 10 is coupled to tissue-stimulating electrodes 16 via one or more electrode leads 18 or 19, which are shown in more details in FIGS. 1B and 1C.

FIG. 1B shows a lead 18 having eight ring-shaped electrodes 16 which are located at different longitudinal positions along a central axis 15. Lead 18 is referred to herein as a “non-directional lead,” because the ring-shaped electrodes span 360 degrees around the axis 15, and thus cannot direct stimulation to different rotational positions around the axis 15.

FIG. 1C shows a lead 19 also having eight electrodes, but not all of the electrodes are ring-shaped. Electrode E8 at the distal end of the lead 19 and electrode E1 at a proximal end of the lead are ring-shaped. Electrodes E2, E3, and E4, by contrast, comprise split-ring electrodes, each of which are located at the same longitudinal position along the axis 15, but each spanning less than 360 degrees around the axis. For example, each of electrodes E2, E3, and E4 may span 90 degrees around the axis 15, with each being separated from the others by gaps of 30 degrees. Electrodes E5, E6, and E7 also comprise split-ring electrodes, but are located at a different longitudinal position. Lead 19 is referred to herein as a “directional lead,” because at least some of the electrodes at a given longitudinal position (e.g., E2, E3, E4) span less than 360 degrees, meaning that those electrodes can direct stimulation to different rotational positions (and hence different brain tissues) around the axis 15. In other designs of a directional lead 19, all electrodes can be split-ring, or there could be different numbers of split-ring electrodes at each longitudinal position (i.e., more or less than three).

Lead wires 20 within the leads are coupled to the electrodes 16 and to proximal contacts 21 insertable into lead connectors 22 fixed in a header 23 on the IPG 10, which header can comprise an epoxy for example. Alternatively, the proximal contacts 21 may connect to lead extensions (not shown) which are in turn inserted into the lead connectors 22. Once inserted, the proximal contacts 21 connect to header contacts 24 within the lead connectors 22, which are in turn coupled by feedthrough pins 25 through a case feedthrough 26 to stimulation circuitry 28 within the case 12, which stimulation circuitry 28 is described below.

In the IPG 10 illustrated in FIG. 1A, there are thirty-two electrodes (E1-E32), split between four percutaneous leads 18 or 19 (18 is shown), and thus the header 23 may include a 2×2 array of eight-electrode lead connectors 22. However, the type and number of leads, and the number of electrodes, in an IPG is application specific and therefore can vary. In another example not shown, a given lead can have 16 sixteen electrodes, and thus this lead would have two sets of proximal contacts 21 to mate with two of the eight-electrode lead connectors 22, as disclosed for example in U.S. Patent Application Publication 2019/0076645. The conductive case 12 can also comprise an electrode (Ec).

In a DBS application, as is useful in the treatment of tremor in Parkinson's disease for example, the IPG 10 is typically implanted under the patient's clavicle (collarbone). Leads 18 or 19 (perhaps as extended by lead extensions, not shown) are tunneled through and under the neck and the scalp, with the electrodes 16 implanted through holes drilled in the skull and positioned for example in the subthalamic nucleus (STN) and the pedunculopontine nucleus (PPN) in each brain hemisphere. The IPG 10 can also be implanted underneath the scalp closer to the location of the electrodes' implantation, as disclosed for example in U.S. Pat. No. 10,576,292. The IPG lead(s) 18 or 19 can be integrated with and permanently connected to the IPG 10 in other solutions.

IPG 10 can include an antenna 27a allowing it to communicate bi-directionally with a number of external devices and systems discussed subsequently. Antenna 27a as shown comprises a conductive coil within the case 12, although the coil antenna 27a can also appear in the header 23. When antenna 27a is configured as a coil, communication with external systems preferably occurs using near-field magnetic induction. IPG 10 may also include a Radio-Frequency (RF) antenna 27b. In FIG. 1A, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Zigbee, WiFi, MICS, and the like. If the IPG 10 lacks a battery 14, an additional coil can be present to receive wireless power from an external source.

Stimulation in IPG 10 is typically provided by pulses each of which may include a number of phases such as 30a and 30b, as shown in the example of FIGS. 2A and 2B. In the example shown, such stimulation is monopolar, meaning that a current is provided between at least one selected lead-based electrode (e.g., E1) and the case electrode Ec 12. Stimulation could be bipolar, in which a current is provided between at least two lead-based electrodes. Stimulation parameters typically include amplitude (current I, although a voltage amplitude V can also be used); frequency (F); pulse width (PW) of the pulses or of its individual phases such as 30a and 30b; the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode E1 has been selected as a cathode (during its first phase 30a), and thus provides pulses which sink a negative current of amplitude −I from the tissue. The case electrode Ec has been selected as an anode (again during first phase 30a), and thus provides pulses which source a corresponding positive current of amplitude +I to the tissue. Note that at any time the current sunk from the tissue (e.g., −I at E1 during phase 30a) equals the current sourced to the tissue (e.g., +I at Ec during phase 30a). The polarity of the currents at these electrodes can be changed: for example, during first phase 30a, Ec can be selected as a cathode, and E1 can be selected as an anode, etc.

IPG 10 as mentioned includes stimulation circuitry 28 to form prescribed stimulation at a patient's tissue. FIG. 3 shows an example of stimulation circuitry 28, which includes one or more current sources 40ᵢ and one or more current sinks 42ᵢ. The sources and sinks 40ᵢ and 42ᵢ can comprise Digital-to-Analog converters (DACs), and may be referred to as PDACs 40ᵢ and NDACs 42ᵢ in accordance with the Positive (sourced, anodic) and Negative (sunk, cathodic) currents they respectively issue. In the example shown, a NDAC/PDAC 40ᵢ/42ᵢ pair is dedicated (hardwired) to a particular electrode node ei 39. Each electrode node ei 39 is connected to an electrode Ei 16 via a DC-blocking capacitor Ci 38, for the reasons explained below. PDACs 40ᵢ and NDACs 42ᵢ can also comprise voltage sources.

Proper control of the PDACs 40ᵢ and NDACs 42ᵢ allows any of the electrodes 16 and the case electrode Ec 12 to act as anodes or cathodes to create a current (such as the pulses described earlier) through a patient's tissue, Z, hopefully with good therapeutic effect. In the example shown, and consistent with the first pulse phase 30a of FIG. 2A, electrode E1 has been selected as a cathode electrode to sink current from the tissue Z and case electrode Ec has been selected as an anode electrode to source current to the tissue Z. Thus PDAC 40_{C} and NDAC 42₁ are activated and digitally programmed to produce the desired current, I, with the correct timing (e.g., in accordance with the prescribed frequency F and pulse width PW). Power for the stimulation circuitry 28 is provided by a compliance voltage VH, as described in further detail in U.S. Patent Application Publication 2013/0289665. Other stimulation circuitries 28 can also be used in the IPG 10. In an example not shown, a switching matrix can intervene between the one or more PDACs 40ᵢ and the electrode nodes ei 39, and between the one or more NDACs 42ᵢ and the electrode nodes. Switching matrices allows one or more of the PDACs or one or more of the NDACs to be connected to one or more electrode nodes at a given time. Various examples of stimulation circuitries can be found in U.S. Pat. Nos. 6,181,969, 8,606,362, 8,620,436, U.S. Patent Application Publications 2018/0071520 and 2019/0083796.

Much of the stimulation circuitry 28 of FIG. 3, including the PDACs 40ᵢ and NDACs 42ᵢ, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as telemetry circuitry (for interfacing off chip with telemetry antennas 27a and/or 27b), circuitry for generating the compliance voltage VH, various measurement circuits, etc.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861.

Referring again to FIG. 2A, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38, as is well known. FIG. 3 also shows that stimulation circuitry 28 can include passive recovery switches 41ᵢ, which are described further in U.S. Patent Application Publications 2018/0071527 and 2018/0140831. Passive recovery switches 41ᵢ may be closed to passively recover any charge remaining on the DC-blocking capacitors Ci 38 after issuance of the second pulse phase 30b—i.e., to recover charge without actively driving a current using the DAC circuitry, as shown during duration 30c. Alternatively, passive charge recovery can be used during the second pulse phase 30b after the actively driven first pulse phase 30a, although this isn't shown in FIG. 2A. Again, passive charge recovery is well known and not further described.

FIG. 4 shows various external systems 60, 70, and 80 that can wirelessly communicate data with the IPG 10. Such systems can be used to wirelessly transmit a stimulation program to the IPG 10—that is, to program its stimulation circuitry 28 to produce stimulation with desired amplitudes and timings as described earlier. Such systems may also be used to adjust one or more stimulation parameters of a stimulation program that the IPG 10 is currently executing, and/or to wirelessly receive information from the IPG 10, such as various status information and measurements, etc.

External controller 60 can be as described in U.S. Patent Application Publication 2015/0080982 for example, and may comprise a portable, hand-held controller dedicated to work with the IPG 10. External controller 60 may also comprise a general-purpose mobile electronics device such as a mobile phone which has been programmed with a Medical Device Application (MDA) allowing it to work as a wireless controller for the IPG 10, as described in U.S. Patent Application Publication 2015/0231402. External controller 60 includes a display 61 and a means for entering commands, such as buttons 62 or selectable graphical icons provided on the display 61. The external controller 60′s user interface enables a patient to adjust stimulation parameters, although it may have limited functionality when compared to systems 70 and 80, described shortly. The external controller 60 can have one or more antennas capable of communicating with a compatible antenna in the IPG 10, such as a near-field magnetic-induction coil antenna 64a and/or a far-field RF antenna 64b.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 4, the computing device is shown as a laptop computer that includes typical computer user interface means such as a display 71, buttons 72, as well as other user-interface devices such as a mouse, a keyboard, speakers, a stylus, a printer, etc., not all of which are shown for convenience. Also shown in FIG. 4 are accessory devices for the clinician programmer 70 that are usually specific to its operation as a stimulation controller. A communication “wand” 76 coupleable to suitable ports on the computing device can include an IPG-compliant antenna such as a coil antenna 74a or an RF antenna 74b. The computing device itself may also include one or more RF antenna 74b. The clinician programmer 70 can also communicate with other devices and networks, such as the Internet, either wirelessly or via a wired link provided at an Ethernet or network port.

External system 80 comprises another means of communicating with and controlling the IPG 10 via a network 85 which can include the Internet. The network 85 can include a server 86 programmed with IPG communication and control functionality, and may include other communication networks or links such as WiFi, cellular or land-line phone links, etc. The network 85 ultimately connects to an intermediary device 82 having antennas suitable for communication with the IPG's antenna, such as a near-field magnetic-induction coil antenna 84a and/or a far-field RF antenna 84b. Intermediary device 82 may be located generally proximate to the IPG 10. Network 85 can be accessed by any user terminal 87, which typically comprises a computer device associated with a display 88. External system 80 allows a remote user at terminal 87 to communicate with and control the IPG 10 via the intermediary device 82.

FIG. 4 also shows circuitry 90 involved in any of external systems 60, 70, or 80. Such circuitry can include control circuitry 92, which can comprise any number of devices such as one or more microprocessors, microcomputers, FPGAs, DSPs, other digital logic structures, etc., which are capable of executing programs in a computing device. Such control circuitry 92 may contain or coupled with memory 94 which can store external system software 96 for controlling and communicating with the IPG 10, and for rendering a Graphical User Interface (GUI) 99 on a display (61, 71, 88) associated with the external system. In external system 80, the external system software 96 would likely reside in the server 86, while the control circuitry 92 could be present in either or both the server 86 or the terminal 87.

### SUMMARY

A method is disclosed for optimizing stimulation for a patient having a stimulator device, wherein the stimulator device comprises a lead with a plurality of electrodes for providing stimulation, the method providing test stimulation at a plurality of different combinations of a position on the lead and a value of at least one stimulation parameter. The method may comprise: (a) providing test stimulation at initial of the combinations, and measuring a neural potential in response to the test stimulation for each of the initial combinations; (b) determining at least one score for each of the initial combinations, wherein one of the at least one scores comprises a neural response score based on a feature of the measured neural potential; (c) determining a next combination using at least all previously determined neural response scores; (d) repeating the steps prescribed in steps (a)-(c) for the next combination to determine and test further next combinations until a stopping criterium is met; and (e) using at least the neural response scores to determine optimal therapeutic stimulation for the patient.

In one example, each measured neural potential comprises an Evoked Resonant Neural Response (ERNA). In one example, the lead is implanted in a brain of the patient. In one example, the at least one stimulation parameter comprises stimulation amplitude. In one example, neural potentials are measured in step (a) using at least one of the electrodes. In one example, the positions vary longitudinally or rotationally, or both, about the lead. In one example, determining the optimal therapeutic stimulation in step (e) comprises determining an optimal combination of a position and a value of the at least one stimulation parameter. In one example, determining the next combination in step (c) comprises determining at least one factor at each possible combination of positions and values of the at least one stimulation parameter, wherein the at least one factor at each possible combination is computed using all previously determined the neural response scores. In one example, the at least one factor at each possible combination is further computed using a distance between each possible combination and each of the previously tested combinations. In one example, the next combination is determined in step (c) as the possible combination having the best at least one factor. In one example, a plurality of factors are determined at each possible combination, and wherein the factors are weighted to determine a weighted factor at each possible combination. In one example, the next combination is determined in step (c) using the weighted factors. In one example, the next combination is determined in step (c) as the possible combination having the best weighted factor. In one example, a second score is additionally determined for each tested combination in step (b). In one example, the second score is indicative of a patient symptom, a patient response, or a side effect in response to the test stimulation. In one example, in step (c) the next combination is determined using all previously determined neural response scores and all previously determined second scores. In one example, in step (e) the optimal combination is determined using the neural response scores and the second scores.

A system is disclosed, which may comprise: a stimulator device comprising a lead with a plurality of electrodes for providing stimulation; and an external device for optimizing stimulation for a patient having the stimulator device, the external device configured to communicate with the stimulator device to provide test stimulation at a plurality of different combinations of a position on the lead and a value of at least one stimulation parameter, wherein the external device is configured to: (a) cause the stimulator device to provide test stimulation at initial of the combinations, and to measure a neural potential in response to the test stimulation for each of the initial combinations; (b) determine at least one score for each of the initial combinations, wherein one of the at least one scores comprises a neural response score based on a feature of the measured neural potential; (c) determine a next combination using at least all previously determined neural response scores; (d) repeat the steps prescribed in steps (a)-(c) for the next combination to determine and test further next combinations until a stopping criterium is met; and (e) use at least the neural response scores to determine optimal therapeutic stimulation for the patient.

In one example, each measured neural potential comprises an Evoked Resonant Neural Response (ERNA). In one example, the lead is configured to be implanted in a brain of the patient. In one example, the at least one stimulation parameter comprises stimulation amplitude. In one example, neural potentials are measured in step (a) using at least one of the electrodes. In one example, the positions vary longitudinally or rotationally, or both, about the lead. In one example, the optimal therapeutic stimulation in step (e) comprises determining an optimal combination of a position and a value of the at least one stimulation parameter. In one example, determining the next combination in step (c) comprises determining at least one factor at each possible combination of positions and values of the at least one stimulation parameter, wherein the at least one factor at each possible combination is computed using all previously determined the neural response scores. In one example, the at least one factor at each possible combination is further computed using a distance between each possible combination and each of the previously tested combinations. In one example, the next combination is determined in step (c) as the possible combination having the best at least one factor. In one example, a plurality of factors are determined at each possible combination, and wherein the factors are weighted to determine a weighted factor at each possible combination. In one example, the next combination is determined in step (c) using the weighted factors. In one example, the next combination is determined in step (c) as the possible combination having the best weighted factor. In one example, a second score is additionally determined for each tested combination in step (b). In one example, the second score is indicative of a patient symptom, a patient response, or a side effect in response to the test stimulation. In one example, in step (c) the next combination is determined using all previously determined neural response scores and all previously determined second scores. In one example, in step (e) the optimal combination is determined using the neural response scores and the second scores.

A method is disclosed for optimizing stimulation for a patient having an implantable stimulator device, wherein the stimulation device comprises a lead with a plurality of electrodes for providing stimulation. The method may comprise: (a) providing test stimulation at a first plurality of combinations of positions on the lead and values of at least one stimulation parameter, and measuring a neural potential in response to the test stimulation for each of the first combinations; (b) using the measured neural potentials to determine excluded positions and/or excluded values of the at least one stimulation parameter; (c) executing an algorithm that iteratively determines a second plurality of combinations of positions on the lead and values of the at least one stimulation parameters at which to provide further test stimulation based at least on first scores resulting from previously-tested of the second combinations, wherein the second combinations do not comprise the excluded positions and/or the excluded values of the at least one stimulation parameter; and (d) using at least the first scores for each of the previously-tested second combinations to determine optimal therapeutic stimulation for the patient.

In one example, the first scores are indicative of an efficacy of the test stimulation for each of the second combinations. In one example, the algorithm further comprises measuring a neural potential in response to the test stimulation for each of the second combinations. In one example, the at least one first score for each second combination is based on a feature of the measured neural potential for each of the second combinations. In one example, the measured neural potentials comprise Evoked Resonant Neural Responses (ERNAs). In one example, the lead is implanted in a brain of the patient. In one example, the at least one stimulation parameter comprises stimulation amplitude. In one example, the neural potentials are measured in step (a) using at least one of the electrodes. In one example, the positions vary longitudinally or rotationally, or both, about the lead. In one example, determining the optimal therapeutic stimulation in step (d) comprises determining an optimal combination of a position and a value of the at least one stimulation parameter. In one example, there is additionally a second score resulting from each of the previously-tested of the second combinations. In one example, the second scores are indicative of a patient symptom, a patient response, or a side effect in response to the test stimulation. In one example, the first scores are determined from a neural potential measured in response to the test stimulation for each of the previously-tested second combinations. In one example, in step (d) the optimal combination is determined using the first scores and the second scores.

A system is disclosed, which may comprise: a stimulator device comprising a lead with a plurality of electrodes for providing stimulation; and an external device for optimizing stimulation for a patient having the stimulator device, the external device configured to communicate with the stimulator device to provide test stimulation at a plurality of different combinations of a position on the lead and a value of at least one stimulation parameter, wherein the external device is configured to (a) cause the stimulator device to provide test stimulation at a first plurality of the combinations, and measure a neural potential in response to the test stimulation for each of the first combinations; (b) use the measured neural potentials to determine excluded positions and/or excluded values of the at least one stimulation parameter; (c) execute an algorithm configured to iteratively determine a second plurality of the combinations at which to provide further test stimulation based at least on first scores resulting from previously-tested of the second combinations, wherein the second combinations do not comprise the excluded positions and/or the excluded values of the at least one stimulation parameter; and (d) use at least the first scores for each of the previously-tested second combinations to determine optimal therapeutic stimulation for the patient.

In one example, the first scores are indicative of an efficacy of the test stimulation for each of the second combinations. In one example, the algorithm is further configured to measure a neural potential in response to the test stimulation for each of the second combinations. In one example, the at least one first score for each second combination is based on a feature of the measured neural potential for each of the second combinations. In one example, the measured neural potentials comprise Evoked Resonant Neural Responses (ERNAs). In one example, the lead is configured to be implanted in a brain of the patient. In one example, the at least one stimulation parameter comprises stimulation amplitude. In one example, the neural potentials are measured in step (a) using at least one of the electrodes. In one example, the positions vary longitudinally or rotationally, or both, about the lead. In one example, determining the optimal therapeutic stimulation in step (d) comprises determining an optimal combination of a position and a value of the at least one stimulation parameter. In one example, there is additionally a second score resulting from each of the previously-tested of the second combinations. In one example, the second scores are indicative of a patient symptom, a patient response, or a side effect in response to the test stimulation. In one example, the first scores are determined from a neural potential measured in response to the test stimulation for each of the previously-tested second combinations. In one example, in step (d) the optimal combination is determined using the first scores and the second scores.

### BRIEF DESCRIPTION OF THE DRAWINGS

FIG. 1A shows an Implantable Pulse Generator (IPG), in accordance with the prior art.

FIG. 1B shows a percutaneous lead having ring electrodes, and FIG. 1C shows a percutaneous lead having split ring electrodes, in accordance with the prior art.

FIGS. 2A and 2B show an example of stimulation pulses (waveforms) producible by the IPG, in accordance with the prior art.

FIG. 3 shows an example of stimulation circuitry useable in the IPG, in accordance with the prior art.

FIG. 4 shows various external systems capable of communicating with and programming stimulation in an IPG, in accordance with the prior art.

FIG. 5A shows a Graphical User Interface (GUI) operable on an external system such as a clinician programmer, which is capable of programming a stimulation program for the IPG.

FIG. 5B shows waveforms produced at the electrodes through use of the GUI of FIG. 5A.

FIG. 6 shows a neural potential, specifically an Evoked Resonant Neural Response (ERNA) produced in response to stimulation in a Deep Brain Stimulation (DBS) system.

FIG. 7 shows a DBS IPG with neural response sensing capability.

FIG. 8 shows a neural potential measurement algorithm used to determine an optimal position on a lead to provide stimulation for a DBS patient using sensed ERNAs.

FIG. 9 shows how the neural potential measurement algorithm can determine extracted features for potentials sensed at various electrodes, and how the values of those features can be processed and scored to determine an optimal position for stimulation.

FIG. 10 shows a programming algorithm for optimizing stimulation in a patient, including the ability to determine an optimal longitudinal position and rotational angle of the stimulation, as well as an optimal amplitude.

FIG. 11 shows an example of a GUI representation of a lead for which stimulation is optimized, and further shows parameters spaces useful to understanding the algorithm.

FIG. 12 shows steps involving how an optimal longitudinal position (Lopt) and an optimal amplitude (Iopt1) are determined in a first part of the algorithm.

FIG. 13 shows how various potential (L, I) values can be excluded using measured neural responses (e.g., ERNAs).

FIG. 14 shows steps in the algorithm relevant to selecting a next (L, I) value to be tested.

FIGS. 15A-15F show sub-steps involved in the steps shown in FIGS. 8A and 8B.

FIG. 16 shows steps involving how an optimal rotational angle (θopt) and an optimal amplitude (Iopt2) are determined at Lopt in a second part of the algorithm.

FIG. 17 shows the GUI that may be used to implement the programming algorithm, and to display information that may be useful to the clinician as the algorithm operates.

FIG. 18 shows a modification to the algorithm in which the clinician can enter a plurality of scores for each tested stimulation parameter set at each iteration of the algorithm.

### DETAILED DESCRIPTION

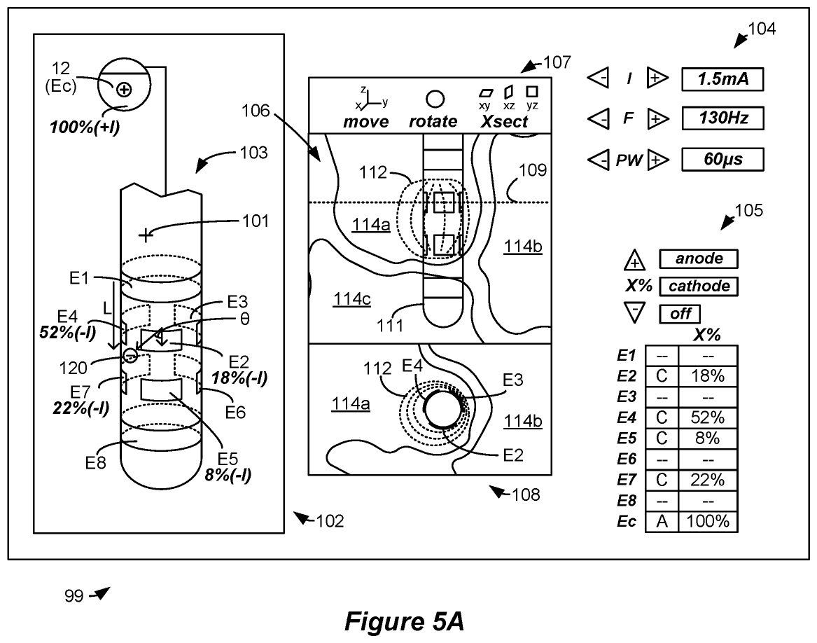

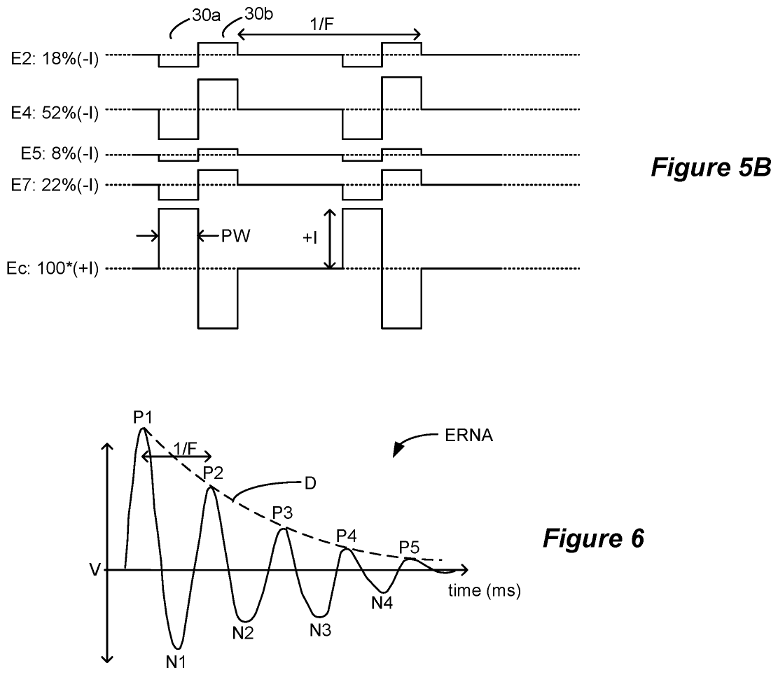

FIG. 5A shows an example of GUI 99 renderable on the display of an external system, such as the clinician programmer 70 mentioned earlier. GUI 99 is particularly useful in an DBS context because it provides a clinician with a visual indication of how stimulation selected for a patient will interact with the brain tissue in which the electrodes are implanted. GUI 99 can be used during surgical implantation of the leads 18 or 19 and its IPG 10, but can also be used after implantation to assist in selecting a therapeutically useful stimulation program for the patient. The GUI 99 can be controlled by a cursor 101 that the user can move using a mouse connected to the clinician programmer 70 for example.

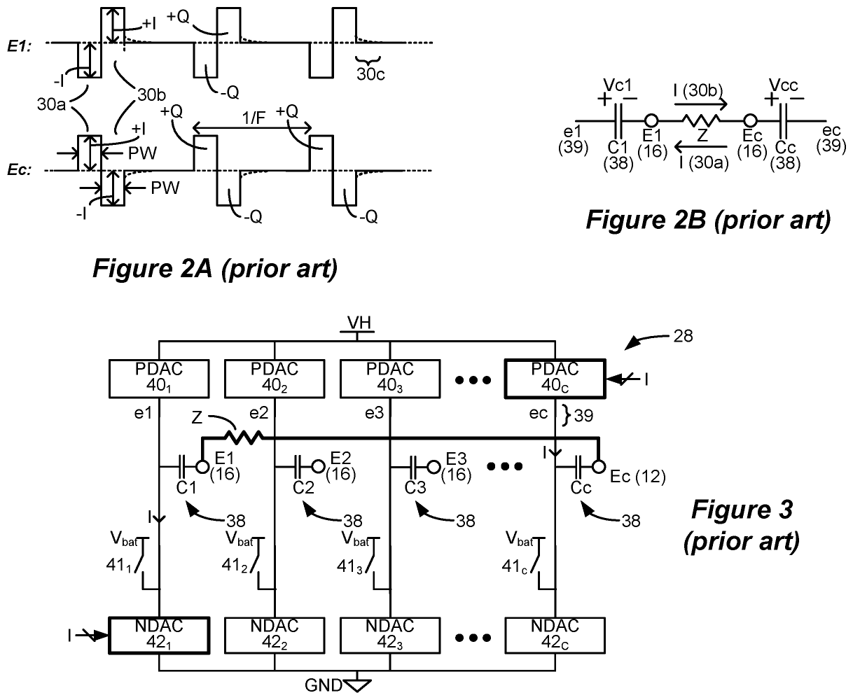

The GUI 99 may include a waveform interface 104 where various aspects of the stimulation can be selected or adjusted. For example, waveform interface 104 allows a user to select an amplitude (e.g., a current I), a frequency (F), and a pulse width (PW) of the stimulation pulses. Waveform interface 104 can be significantly more complicated, particularly if the IPG 10 supports the provision of stimulation that is more complicated than a repeating sequence of pulses. Waveform interface 104 may also include inputs to allow a user to select whether stimulation will be provided using biphasic (FIG. 2A) or monophasic pulses, or in bursts of pulses, and to select whether passive charge recovery will be used, although again these details aren't shown for simplicity.

The GUI 99 may also include an electrode configuration interface 105 which allows the user to select a particular electrode configuration specifying which electrodes should be active to provide the stimulation, and with which polarities and relative magnitudes. In this example, the electrode configuration interface 105 allows the user to select whether an electrode should comprise an anode (A) or cathode (C) or be off, and allows the amount of the total anodic or cathodic current +I or −I (specified in the waveform interface 104) that each selected electrode will receive to be specified in terms of a percentage, X. For example, in FIG. 5A, the case electrode 12 Ec is specified to be an anode that receives X=100% of the current I as an anodic current +I (e.g., during first pulse phase 30a if biphasic pulses are used; see FIG. 2A). The corresponding cathodic current −I is split between cathodes electrodes E2 (18% or 0.18*−I), E4 (52% or 0.52*−I), E5 (8% or 0.08*−I), and E7 (22% or 0.22*−I) (again during first pulse phase 30a). The waveforms resulting at the electrodes from this electrode configuration are shown in FIG. 5B. Note that two or more electrodes can be chosen to act as anodes or cathodes at a given time, allowing the electric field in the tissue to be shaped, as explained further below. Once the waveform parameters (104) and electrode configuration parameters (105) are determined, they can be sent from the clinician programmer 70 to the IPG 10, so that the IPG's stimulation circuitry 28 (FIG. 3) can be programmed (the various NDACs and PDACs) to produce the desired currents at the selected electrodes with the proper timing. For example, PDAC 40c would be programmed to produce +100%*+I, and NDAC 424 would be programmed to produce 52%*−I, etc. Together, the various waveform parameters and electrode configuration parameter comprise stimulation parameters, which together comprise a stimulation program.

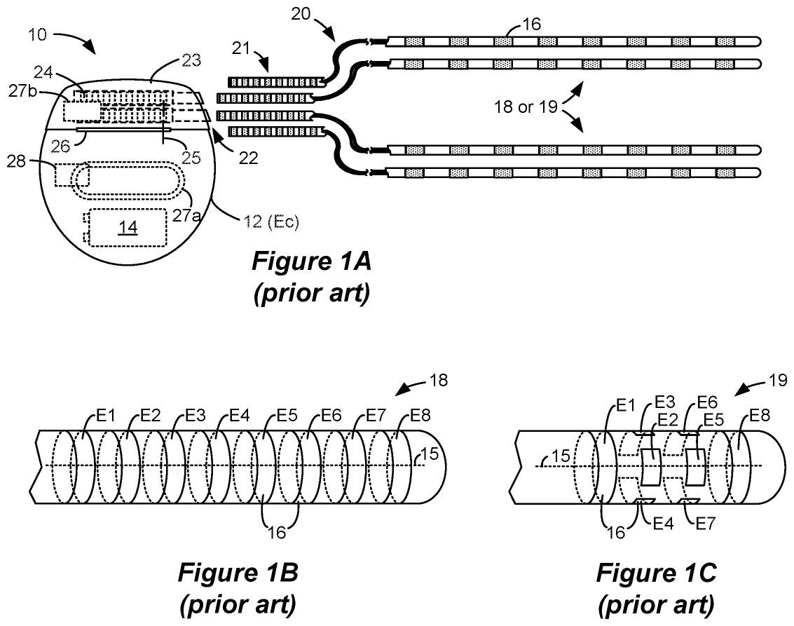

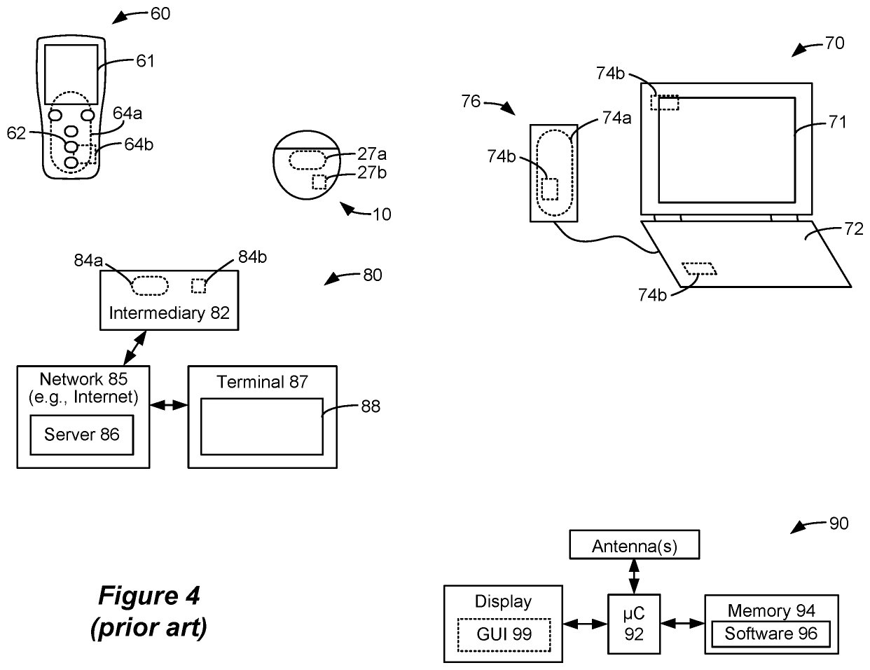

Use of these electrodes to provide cathodic stimulation sets a particular position for a cathodic pole 120 in three-dimensional space. The position of this cathode pole 120 can be quantified at a particular longitudinal position L along the lead (e.g., relative to a point on the lead such as the longitudinal position of electrode E1), and at a particular rotational angle θ (e.g., relative to a particular angle on the lead such as relative to the center of electrode E2). (Note that rotation angle θ is only relevant when a directional lead such as 19 (FIG. 1C) is used). This position is shown in a leads interface 102 of the GUI 99. Notice that the position of the pole 120 (L,θ) may be virtual; that is, the position may not necessarily occur at the physical position of any of the electrodes 16 in the electrode array, as explained further later. The leads interface 102 preferably also includes an image 103 of the lead being used for the patient. Although not shown, the leads interface 102 can include a selection to access a library of relevant representations 103 of the types of leads (e.g., 18 or 19) that may be implanted in different patients, which may be stored with the relevant software (e.g., 96, FIG. 4). The cursor 101 can be used to select an illustrated electrode 16 (e.g., E1-E8, or the case electrode Ec), or a pole such as cathode pole 120. Pole 120 could also be anodic, or there could be more than one pole if multipolar stimulation is used, but this isn't shown.

An electrode configuration algorithm (not shown), operating as part of external device's software 96, can determine a position of the cathode pole 120 in three-dimensional space from a given electrode configuration, and can also conversely determine an electrode configuration from a given position of the pole 120. For example, the user can place the position of the pole 120 using the cursor 101. The electrode configuration algorithm can then be used to compute an electrode configuration that best places the pole 120 in this position. Note that cathode pole 120 is positioned closest to electrode E4, but is also generally proximate to electrodes E2, E7, and E6. The electrode configuration algorithm may thus calculate that electrode E4 should receive the largest share of cathodic current (52%*−I), while E2, E7, and E6 which are farther away from the pole 120 receive lesser percentages, as shown in the stimulation parameters interface 104. By involving more than one electrode, cathode pole 120 is formed as a virtual pole not as the position of any of the physical electrodes. Again, the electrode configuration algorithm can also operate in reverse: from a given electrode configuration, the position of the pole 120 can be determined. The electrode configuration algorithm is described further in U.S. Patent Application Publication 2019/0175915, which is incorporated herein by reference.

GUI 99 can further include a visualization interface 106 that allows a user to view a stimulation field image 112 formed on a lead given the selected stimulation parameters and electrode configuration. The stimulation field image 112 is formed by field modelling in the clinician programmer 70, as discussed further in the '091 Publication. The visualization interface 106 preferably, but not necessarily, further includes tissue imaging information 114. This tissue imaging information 114 is presented in FIG. 5A as three different tissue structures 114a, 114b and 114c in FIG. 6 for the patient in question, which tissue structures may comprise different areas of the brain for example. Such tissue imaging information may come from a Magnetic Resonance Image (MRI) or Computed Tomography (CT) image of the patient, may come from a generic library of images, and may include user defined regions. The GUI 99 can overlay the lead image 111 and the stimulation field image 112 with the tissue imaging information 114 in the visualization interface 106 so that the position of the stimulation field 112 relative to the various tissue structures 114i can be visualized. The various images shown in the visualization interface 106 (i.e., the lead image 111, the stimulation field image 112, and the tissue structures 114i) can be three-dimensional in nature, and hence may be rendered to allow such three-dimensionality to be better appreciated by the user, such as by shading or coloring the images, etc. A view adjustment interface 107 may allow the user to move or rotate the images, using cursor 101 for example, as explained in the '091 Publication. In FIG. 5A, a cross-section interface 108 allows the various images to be seen in a particular two-dimensional cross section, and in this example a cross section 109 is shown taken perpendicularly to the lead image 111 and through split-ring electrodes E2, E3, and E4. Interfaces 106 and 108 may also show the cathode pole 120 in a proper position, but this isn't shown.

The GUI 99 of FIG. 5A is particularly useful because it allows the electric field as reflected in stimulation field image 112 (or the pole 120) to be seen relative to surrounding tissue structures 114i. This allows the user to adjust the stimulation parameters to recruit, or avoid recruiting, particular tissue structures 114i. Assume for example that it is desirable for a given patient to stimulate tissue structure 114a, but to not stimulate tissue structures 114b or 114c. This may be because tissue structure 114a is causing undesired patient symptoms (e.g., tremor) that stimulation can alleviate, while stimulation of tissue structures 114b and 114c will cause undesired side effects. The clinician can then use GUI 99 to adjust stimulation (e.g., to adjust the stimulation parameters or the electrode configuration) to move the stimulation field 112 (e.g., the cathode pole 120) to a proper position (L, θ). In the example shown, and as best seen in the cross-section interface 108, higher cathodic currents are provided at split-ring electrodes E4 (0.52*−I) and E2 (0.18*−I) because these electrodes are generally facing towards tissue structure 114a which should be stimulated. By contrast, split-ring electrode E3 carries no cathodic current because it generally faces towards tissue structure 114b where stimulation is ideally avoided. The result is a stimulation field 112 that is more predominant in tissue structure 114a and less predominant in tissue structure 114b, as shown in the visualization interface 106.

Especially in a DBS application, it is important that correct stimulation parameters be determined for a given patient. Improper stimulation parameters may not yield effective relief of a patient's symptoms, or may cause unwanted side effects. To determine proper stimulation, a clinician typically uses GUI 99 to try different combinations of stimulation parameters. This may occur, at least in part, during a DBS patient's surgery when the leads are being implanted. Such intra-operative determination of stimulation parameters can be useful to determine a general efficacy of DBS therapy. However, finalizing stimulation parameters that are appropriate for a given DBS patient typically occurs after surgery after the patient has had a chance to heal, and after the position of the leads stabilize in the patient. Thus, at such time, the patient will typically present to the clinician's office to determine (or further refine) optimal stimulation parameters during a programming session.

Gauging the effectiveness of a given set of stimulation parameters typically involves programming the IPG 10 with that set, and then reviewing the therapeutic effectiveness and side effects that result. Therapeutic effectiveness and side effects are often assessed by one or more different scores (S) for one or more different clinical responses, which are entered into the GUI 99 of the clinician programmer 70 where they are stored with the stimulation parameters set being assessed. Such scores can be subjective in nature, based on patient or clinician observations. For example, bradykinesia (slowness of movement), rigidity, tremor, or other symptoms or side effects, can be scored by the patient, or by the clinician upon observing or questioning the patient. Such scores in one example can range from 0 (best) to 4 (worst).

Scores can also be objective in nature based on measurements taken regarding a patient's symptoms or side effects. For example, a Parkinson's patient may be fitted with a wearable sensor that measures tremors, such as by measuring the frequency and amplitude of such tremors. A wearable sensor may communicate such metrics back to the GUI 99, and if necessary, converted to a score. U.S. Patent Application Publication 2021/0196956, which is incorporated herein by reference in its entirety, discusses determining which symptoms and/or side effects are most sensible to score for a given patient when the stimulation parameters are optimized.

One type of objective measurement proposed for use in DBS systems are Evoked Resonant Neural Activity (ERNA) responses, which are described in U.S. patent application Ser. No. 17/932,928, filed Sep. 16, 2022. See, e.g., Sinclair, et al., “Subthalamic Nucleus Deep Brain Stimulation Evokes Resonant Neural Activity,” Ann. Neurol. 83(5), 1027-31 (2018). An example ERNA response to DBS stimulation is shown in FIG. 6. An ERNA comprises a neural response provided by brain tissue in response to stimulation. As shown in FIG. 6, ERNA has an oscillatory voltage response, which comprises a number of positive peaks Pi and negative peaks Ni, which may have characteristic amplitudes, separations, or latencies. These peaks may issue with a frequency F of about 200 to about 500 Hz, and may decay according to a characteristic decay function D, which may be roughly understood or modelled as an exponential decay. Stimulation of the STN, and particularly of the dorsal subregion of the STN, has been observed to evoke strong ERNA responses, whereas stimulation of the posterior subthalamic area (PSA) does not evoke such responses. Thus, ERNA may provide a biomarker for selecting appropriate electrodes for stimulation and for achieving the desired therapeutic response. Having said this, ERNA is just one type of neural potential that can be monitored in response to stimulation. Local field potentials (LFPs), DBS local evoked potentials (DLEPs), evoked compound activity (ECA), single or multi unit activities (SUA/MUA), or even EEG/ECoG may also be monitored and used in the disclosed techniques. Other brain regions which may evoke or not evoke ERNA may also be employed.

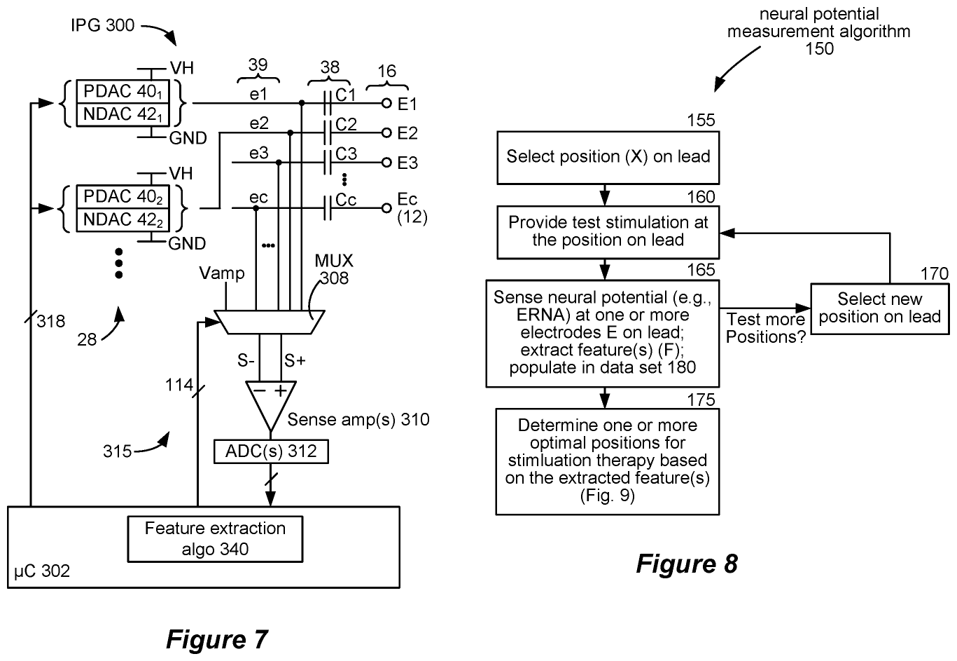

The '928 Application discloses an IPG capable of sensing neural potentials such as ERNAs, and this IPG 300 is shown in FIG. 7. The IPG 300 includes control circuitry 302, which may comprise a microcontroller, such as Part Number MSP430, manufactured by Texas Instruments, Inc., which is described in data sheets at http://www.ti.com/microcontrollers/msp430-ultra-low-power-mcus/overview.html, which are incorporated herein by reference. Other types of controller circuitry may be used in lieu of a microcontroller as well, such as microprocessors, FPGAs, DSPs, or combinations of these, etc. Control circuitry 302 may also be formed in whole or in part in one or more Application Specific Integrated Circuits (ASICs), such as those described and incorporated earlier.

The IPG 300 also includes the stimulation circuitry 28 described earlier (see FIG. 3), and further includes sensing circuitry 315 for sensing neural potentials such as ERNAs. As shown, each electrode node 39 is coupleable to a sense amp circuit 310 via a multiplexer (MUX) 308. The multiplexer is controlled by a bus 314 to allow one or more of the electrodes to be selected as a sensing electrode. Although only one MUX 308 and sense amp circuit 310 are shown in FIG. 7, there could be more than one. For example, there can be four MUX 308/sense amp circuit 310 pairs each operable within one of four timing channels supported by the IPG 300 to provide stimulation. The sensed signals output by the sense amp circuitry are preferably converted to digital signals by one or more Analog-to-Digital converters (ADC(s)) 312, which may sample the output of the sense amp circuit 310 at 50 kHz for example. The ADC(s) 312 may also reside within the control circuitry 302, particularly if the control circuitry 302 has A/D inputs. Sensing may be differential, with the MUX 308 passing two electrode nodes to the sense amp circuit 310. As one skilled in the art understands, differential sensing will cancel out any common mode voltage in the tissue from both inputs. Sensing may also be single-ended, with the multiplexer 308 selecting one electrode node and a fixed reference voltage, Vamp. Examples of sense amp circuitry 310, and manner in which such circuitry can be used, can be found in U.S. Patent Application Publications 2019/0299006, 2020/0305744, and 2020/0305745.

Once sensed and digitized, the neural responses can be processed by a feature extraction algorithm 340 programmed as firmware in the control circuitry 302. The feature extraction algorithm 340 can determine one or more features of sensed neural response. As relevant to ERNA measurements, examples of such features include but are not limited to:
a height of any peak (e.g., N1);
a peak-to-peak height between any two peaks (such as from N1 to P2);
a ratio of peak heights (e.g., N1/P2);
a peak width of any peak (e.g., the full-width half-maximum of N1);
an area or energy under any peak;
a total area or energy comprising the area or energy under positive peaks with the area or energy under negative peaks subtracted or added;
a length of any portion of the curve of the evoked potential (e.g., the length of the curve from P1 to N2);
any time defining the duration of at least a portion of the evoked potential (e.g., the time from P1 to N2);
latencies of any peaks (P1, N1, etc.) as well as other feature-to-feature latencies;
amplitude decay function D;
a time delay from stimulation to issuance of the evoked potential, which is indicative of the neural conduction speed of the evoked potential, which can be different in different types of neural tissues;
a conduction speed (i.e., conduction velocity) of the evoked potential, which can be determined by sensing the evoked potential as it moves past different sensing electrodes;
a rate of variation of any of the previous features, i.e., how such features change over time;
a power (or energy) determined in a specified frequency band (e.g., delta, alpha, beta, gamma, etc.) determined in a specified time window (for example, a time window that overlaps the neural response, the stimulation artifact, etc.);
spectral characteristics in the frequency domain (e.g., Fourier transform);
a cross-correlation or cross-coherence of the evoked potential shape with a target optimal shape; and
any mathematical combination or function of these features.
   These and other features may be determined on various portions or sub-portions of the recorded neural responses, which may involve various levels of processing.

FIG. 8 shows a neural potential measurement algorithm 150 that can be used to determine good/best electrodes to use for a DBS patient based on measured neural potentials. As noted earlier, various neural potentials can be monitored, such as the neural responses to stimulation noted earlier (e.g., ERNA responses). Algorithm 150 would likely be implemented or automated using an external system such as clinician programmer 70, although the IPG 300 may also perform some of the algorithm's steps, as explained further below. While this and other algorithms are disclosed as working in conjunction with an IPG 300, one skilled will understand that such algorithms can be used if leads have been implanted but the IPG 300 hasn't yet. Such implanted leads may be connected through the tissue to an external device, such as an operating room programmer or an External Trial Stimulator. Use of the algorithms in this context may be desired during experimentation, extended standard-of-care visits, IPG-replacement surgery, etc.

First, an initial position X on the lead is selected (155). This position X may be at a particular longitudinal position along the lead (L), and at a particular rotational position (θ) around the lead, particularly when a directional lead is used (e.g., 19, FIG. 1C). Test stimulation is then provided at position X (160). This test stimulation can for example be provided as a monopole (e.g., cathode) that is located at the position (with the case electrode Ec acting as an anodic return). As explained above, this selected position can be arrived at through use of an electrode configuration algorithm, which may split anodic and cathodic currents appropriately to place the stimulation at position X.

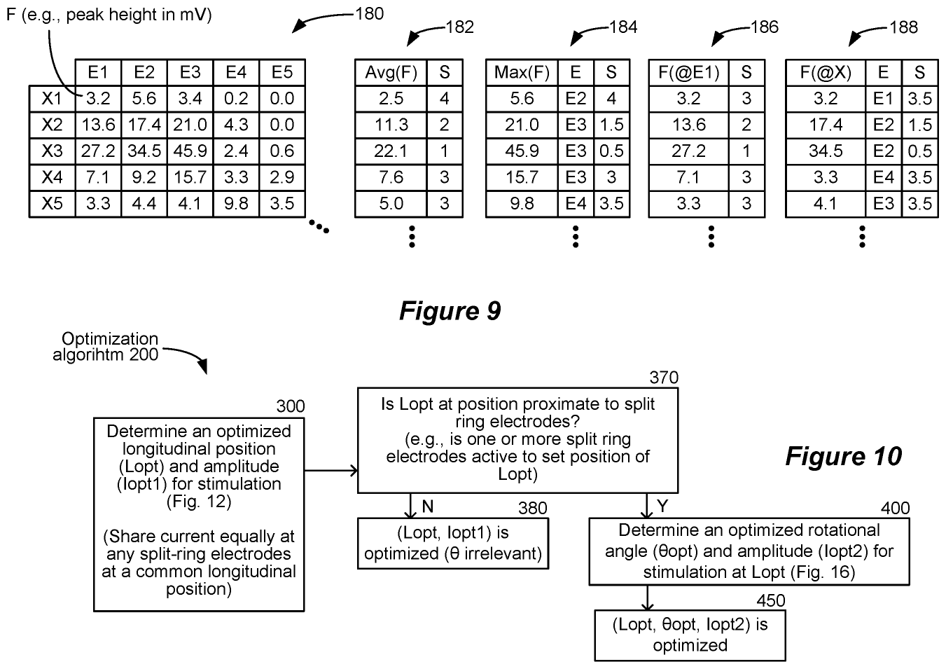

One or multiple neural potentials (e.g., ERNAs) are then sensed in response to the test stimulation (165). In a preferred example, the neural potentials may be measured at a plurality of (or all) electrodes E on the IPG. Conversely, a neural potential may be measured at a single electrode, such as one that is at (or nearest to) position X, or at a suitable distance from position X. One or more features are then extracted for the neural potential measured at each electrode, using feature extraction algorithm 340 in the IPG 300 for example. Potential neural potential features were described earlier, but for simplicity it can be assumed that a maximum peak height (e.g., P1, FIG. 6) comprises the extracted feature. The extracted features may be stored in a data set 180 (FIG. 9), explained further below. This data set 180 may be stored in the IPG 300 (i.e., in conjunction with control circuitry 302), or the extracted features may be transmitted to an external system (e.g., clinician programmer 70) to allow data set 180 to be populated in the external system. The neural potential waveforms can also be transmitted to the external system to allow the features to be extracted and populated in the data set 180 externally.

If it is desired or necessary to test further positions X on the lead, a new position can be selected (170), and test stimulation provided at that new position (160). A new position may be selected following a particular pattern. For example, a position may start at one end of the lead, and may be moved longitudinally or rotationally so that eventually test stimulation is applied at substantially all positions along and around the lead. Neural potentials can again be measured at the new location (165), and still further locations tested (170, 160), etc. In some cases, it may be possible to deliver test stimulation at multiple positions along or about one or more leads partially or wholly simultaneously, in order to accelerate the collection of data.

Once all positions have been selected and tested, neural potential measurement algorithm 150 can then determine one or more optimal positions to apply therapeutic stimulation for the patient based on the extracted features of the measured neural potentials (175). An example of this can occur is shown using data set 180 in FIG. 9, which was populated during execution of the algorithm 150. The data set 180 shows values of a particular extracted feature of (in this example) the ERNA neural responses (F; assumed here to be peak high in mV) as measured at different electrodes E for each position X that was tested during the algorithm. For example, at position X1, a peak height of 3.2 mV is measured at electrode E1, 5.6 mV at E2, etc. These extracted values can be used to determine one or more optimal positions X for stimulation for the patient. Although not illustrated, algorithm 150 can also test different stimulation parameters (e.g., amplitude, pulse width, frequency, etc.) at each stimulation location, and thus neural potential measurements can also be used to determine the efficacy of these stimulation parameters at each position.

Such optimal position(s) may be based of the values of the features, which can be assessed in the algorithm 150 in different ways. For example, the extracted values as sensed at different electrodes can be averaged at each tested position X, as shown in 182. If we assume that higher values for the average indicate best therapeutic results (e.g., 22.1 mV), this would suggest that stimulation at position X3 is best for the patient. Note that averaging is merely one example of how a single value can be determinized for each tested position. Other linear or nonlinear combinations of the values could be used as well.

Alternatively, a maximum of the extracted values at each tested position can be determined, as shown in 184. For example, the maximum feature at position X1 is measured at electrode E2 (5.6 mV); at position X2 is measured at electrode E3 (21.0 mV), etc. If we again assume higher values are preferred (e.g., 45.9 mV), X3 would again be determined as the best position for stimulation.

In another example, the extracted value as sensed at a particular electrode (e.g., electrode E1) can be assessed for all positions, as shown in 186, which would again yield position X3 (27.2 mV) as a best position.

In yet another example, the extracted value at each position X can be sensed at a position relative to X, as shown in 188. This could comprise an electrode that is closest to the position X, or an electrode that is a particular distance from position X, etc. In this example, it is assumed at position X1 that the neural response is best sensed at electrode E1; at positions X2 and X3, the neural response is sensed at electrode E2, possibly because this electrode is closest to both of these positions.

These are simple examples of how extracted features can be used to determine optimal position for stimulation, and the algorithm 150 could operate differently depending on the particulars. For example, for some extracted features, lower values may be preferred. Note that 182-186 can comprise part of data set 180.

While the values of the extracted features can be used in their own right to select one or more optimal positions X for stimulation, such features can also be converted to a score S, similar to the manner in which subjectively-determined effects (e.g., for bradykinesia (slowness of movement), rigidity, tremor, etc.) can be scored to determine best stimulation positions. Thus, the various extracted features in FIG. 9 (182-188) have been assigned a score (e.g., between 0 and 4), with larger (preferred) extracted values being assigned lower (better) scores, and smaller extracted values being assigned higher (worse) scores. Converting the extracted values into scores can assist in processing, as explained further below. Scoring also can be defined in a manner that would reflect a change in the extracted features relative to a baseline (e.g., as determined using non-therapeutic stimulation).

Once the GUI 99 of the clinician programmer 70 has received various scores S (whether subjective or objective, and whether from patient or clinician) for each of the sets of stimulation parameters tested, the clinician may review the scores to try and determine one or more sets of optimal stimulation parameters for the patient which maximize therapeutic effectiveness while minimizing unwanted side effects. Typically, this process involves significant guess work and time, especially when a directional lead (e.g., FIG. 1C) is used. If testing all possible stimulation parameter sets is done as comprehensively as possible, stimulation would need to be provided to the patient at every possible stimulation position (L, θ). Then different combinations of the waveform parameters (e.g., F, PW, and I) would be tested at each of these positions, and then scored (S). In reality, it may only be necessary to optimize the waveform parameter of amplitude (I), as other parameters (F, PW) may be known or determined by other means. Nevertheless, a data set (I, L, θ, S) would need to be determined for all possible combinations of I, L and θ the IPG 10 is capable of producing. This may be a burdensome number of combinations to try during a programming session. At any given combination of I, L and θ, it may take some time (e.g., a matter of minutes) to determine an appropriate score (S). This is particularly true if the scores are based on subjective measurements, because the patient may need to be observed, may need to perform certain tasks (finger tapping, walking, etc.), or may need to answer a number of questions. Because a programming session may only reasonably last a few hours, only a fraction of possible I, L and θ combinations can be tested and scored. While objective measurements such as those based on ERNA neural response measurements may be quicker to perform (FIG. 8), it may still be inefficient to measure such responses at each possible positions X, that is to say at all possible combinations of I, L and θ.

U.S. Patent Application Publication 2022/0257950, which is hereby incorporated by reference in its entirety, discloses an optimization algorithm 200 to efficiently test different I, L and θ combinations with the goal of more quickly arriving at optimal stimulation parameters. Another waveform parameter (e.g., F, PW) could also be optimized along with stimulation positioning data L and θ, but the waveform parameter of amplitude (I) is chosen for optimization given its high significance to patient treatment. Some time is taken to explain algorithm 200 of the '950 Publication, because this algorithm 200 is built upon and supplemented by additional information in this disclosure, as discussed later.

Optimization algorithm 200 is shown at a high level in FIG. 10, with other sub-steps and details shown in subsequent figures. The algorithm 200 preferably first simultaneously determines an optimal longitudinal position (Lopt) and amplitude (Iopt1) for stimulation (300) along the lead. When determining Lopt and Iopt1, and assuming a directional lead (e.g., 19, FIG. 1C) is used, stimulation is positioned symmetrically (non-directionally) around the lead by setting currents equal at split ring electrodes at a common longitudinal position. As explained further below, determining Lopt and Iopt1 involves the algorithm 200 efficiently selecting various values for L and I (L,I) at which stimulation can be tried on the patient and scored. This is an iterative process, and the algorithm 200 automatically determines a next (L,I) value to be tested and scored based on previously tested and scored (L,I) values.

Once Lopt is determined, the algorithm 200 determines whether Lopt is proximate to split ring electrodes (370)—i.e., whether Lopt is longitudinally at or close to split ring electrodes on a directional lead 19. Lopt may be determined to be proximate to split ring electrodes if at least one split ring electrode is used (active) to set the position of Lopt, as explained further below. If Lopt is not proximate to split ring electrodes, as would necessarily be the case when the algorithm 200 is used with a non-directional lead (e.g., 18, FIG. 1B), Lopt and Iopt1 are optimized for the patient (380); optimizing a rotational angle θ is irrelevant and thus not optimized.

If Lopt is proximate to split ring electrodes (370), as might be the case when algorithm 200 is used with a directional lead 19, the algorithm 200 simultaneously determines an optimal rotational angle (θopt) and amplitude (Iopt2) for stimulation around the lead at the optimized longitudinal position Lopt (400). Algorithm 200 may determine that Iopt2 is the same as Iopt1 determined earlier, but it is also likely that Iopt2 will differ from Iopt1 as the rotational angle of stimulation is also optimized. As explained further below, determining θopt and Iopt2 involves the algorithm 200 selecting various values for θ and I (θ,I) which can be tried on the patient and scored. This process can be similar to the manner in which (L,I) values were selected earlier, with the algorithm 200 automatically and efficiently determining next (θ,I) values to be tested and scored based on previously tested and scored (θ,I) values. Once θopt and Iopt2 are optimized, the stimulation is fully optimized for the patient, as the longitudinal position, rotational angle, and amplitude of the stimulation (Lopt, θopt, Iopt2) have now been determined (450).

One skilled in the art will appreciate that programming algorithm 200 can comprise a portion of software 96 operable in the clinician programmer 70 or other external system (FIG. 3). Algorithm 200 can be stored as instructions on a computer-readable medium, such as on a magnetic or optical disk, in solid state memory, etc., and may be so stored in the clinician programmer 70 or in any external system (see FIG. 4).

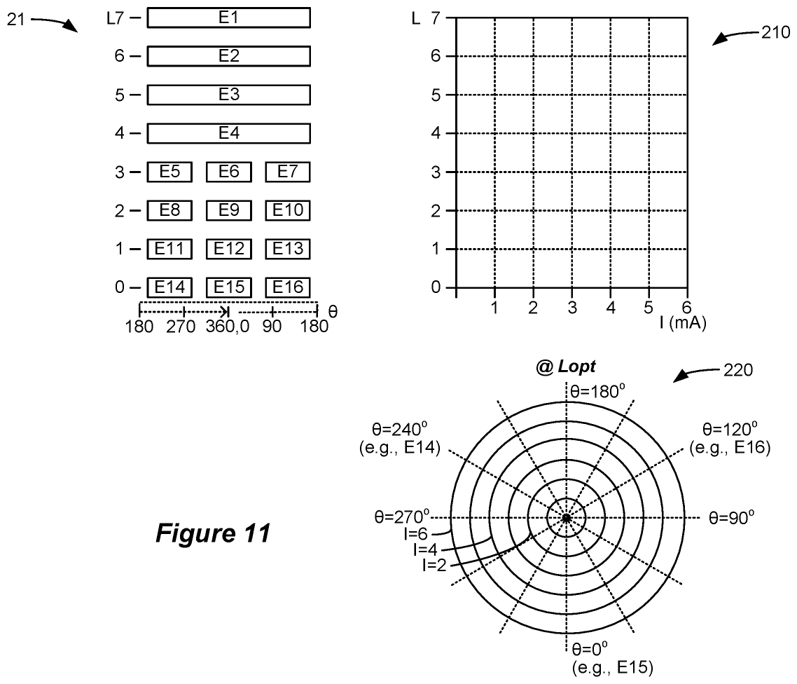

FIG. 11 shows various representations that are useful in understanding the description of programming algorithm 200 that follows. First, a directional lead 21 is illustrated in a two-dimensional manner. A directional lead 21 is shown as a more complicated case, but as noted above algorithm 200 may also be used to determine optimal stimulation parameters when a non-directional lead is used. As shown, this directional lead 21 includes ring electrodes E1-E4 which are respectively located at longitudinal positions (L) 7, 6, 5, and 4, and split ring electrodes E5-E16 which are located at longitudinal positions (L) 3, 2, 1, and 0 as shown. L can also be represented by an actual physical measurement, such as millimeters. Rotational angle θ around the lead 21 is also shown, with 0 degrees being selected at some arbitrary position (such as in the middle of split ring electrodes E6, E9, E12, and E15). Of course, this type of directional lead 21 is just one example, and other directional leads having different combinations of ring and split ring electrodes, or directional leads having split ring electrodes exclusively, could be used as well.

L,I parameter space 210 shows possible values (L,I) that can be tested and optimized, which is particularly useful during step 300 (FIG. 10) when Lopt and Iopt1 are determined. L,I parameter space 210 can have any resolution that the IPG 10 is capable of producing. For example, it can be assumed in one example that the amplitude I of the current is adjustable in increments of 0.1 mA, up to a maximum of 6.0 mA. Longitudinal position L may also be set in increments of tenths.

Parameter space 220 shows possible values for (θ,I) that can be tested and optimized, which is particularly useful during step 400 (FIG. 10, 16) when θopt and Iopt2 are determined at the already-established position of Lopt. θ,I parameter space 220 as shown is circular, with rotational angle θ represented angularly, and with amplitude I represented radially. Parameter space 220 can again have any resolution that the IPG 10 is capable of producing. For example, it can be assumed in one example that the rotational angle θ is adjustable in increments of 30° degrees, and again the amplitude I of the current is adjustable in increments of 0.1 mA up to a maximum of 6.0 mA.

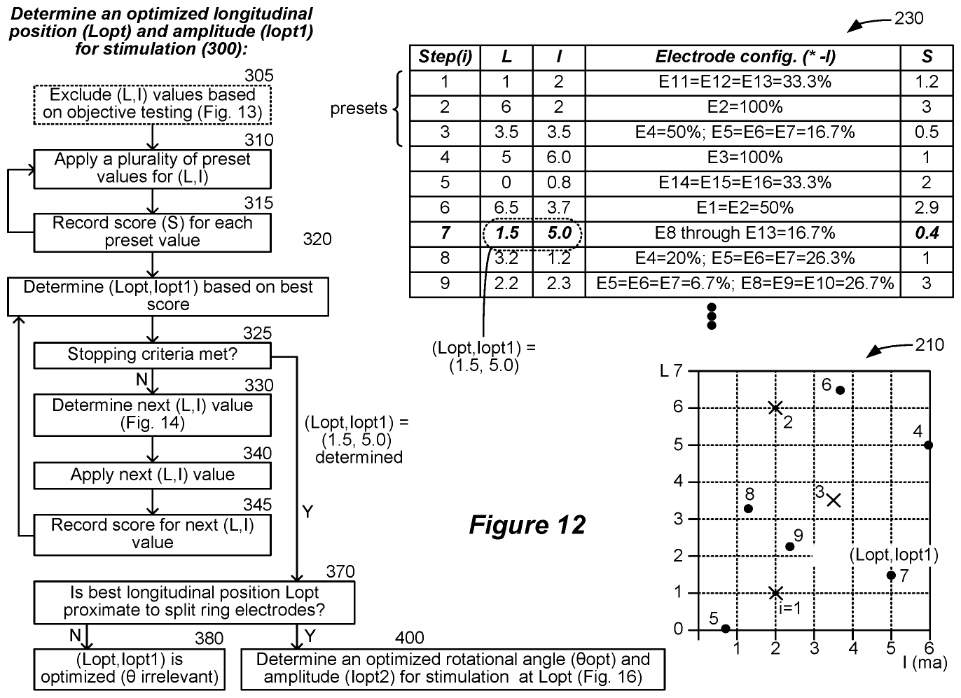

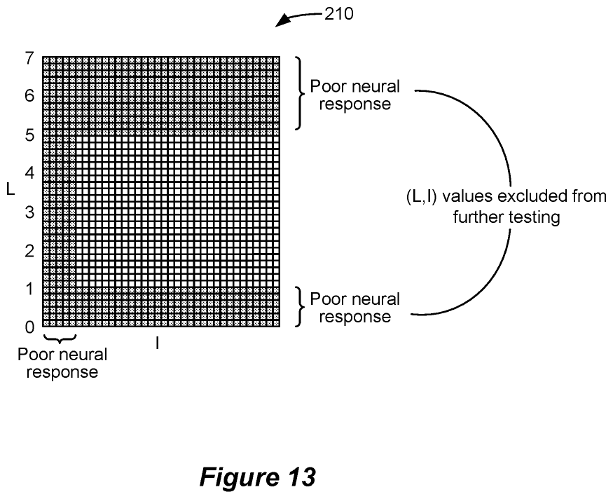

FIG. 12 shows steps involved in determining Lopt and Iopt1 (300). See also U.S. Patent Application Publication 2018/0104500, which is incorporated by reference in its entirety. In a first optional step 305, certain (L,I) values may be excluded from further consideration and testing in the algorithm 200 based on objective testing. In a preferred example, such objective testing can comprise the neural potentials (e.g., ERNA) discussed earlier. In this regard, the neural potential measurement algorithm 150 discussed earlier (FIG. 8) can be run at step 305, or the results from algorithm 150 queried if it was run earlier. As described earlier, algorithm 150 tests various positions X to see which might be optimal for stimulation for a given patient, and in particular can provide extracted values or scores at those positions. Because positions X correspond to different longitudinal positions L, and because testing may occur at one or more known current amplitude I, the positions X correspond to different (L,I) values in L,I parameters space 210, as shown in FIG. 13. If certain (L,I) values were determined to have poor neural potentials (low extracted features values; high scores S), meaning stimulation at that location is unlikely to provided benefit for the patient (e.g., because it is not stimulating the neural target of interest), those values can be excluded from the L,I parameters space 210. In FIG. 13 for example, for certain lead placements, it may be presumed that the neural potentials would be poor at the longitudinal ends of the lead, and thus (L,I) values proximate to these ends are excluded. Thus, zones of L values (e.g., from about 5 to 7, and from about 0 to 1) may be logically excluded, as shown by the grey shading at the top and bottom of the space 210. Likewise, if the algorithm 150 tested different stimulation parameters, and in particular amplitude I, zones of amplitude values may be logically excluded. For example, and as shown in FIG. 13, such earlier objective testing may show that lower amplitudes (I<1 mA) were not yielding suitable neural potentials, and thus are excluded from further testing.

When objective measurements taken at step 305 (e.g., algorithm 150) can be run relatively quickly and passively on the patient, and benefits operation of the algorithm 200 because it allows certain (L,I) values to be ignored as potential optimal stimulation locations for the patient. This speeds up operation of the algorithm 200, and increases the likelihood that algorithm 200 will more quickly and accurately discover optimal stimulation parameters for the patient.

Preferably, the algorithm 200 uses a plurality of preset (L,I) values at its inception (310). These preset values will depend on the type of lead for which stimulation is being optimized, and are preferably chosen to cover a desired portion of the lead to be searched, and/or the most probable position of optimal stimulation. In the example shown, three presets are used with (L,I) values of (1, 2 mA), (6, 2 mA) and (3.5, 3.5 mA), which are sequentially selected in different steps (i=1, 2, and 3) and applied to the patient. As explained further below, applying (L,I) values involves the clinician programmer 70 transmitting stimulation parameter sets (as reflected in the electrode configuration) to the IPG 10 so that stimulation can be produced at the prescribed position (L) and amplitude (I). If one or more of these (L,I) presets has been excluded (305, FIG. 12), it can be relocated by the algorithm 200 to another (L,I) value in the L,I parameter space 210, although this detail is not shown. Furthermore, the presets can be relocated to other (L,I) values depending on the results of objective testing at step 305. For example, it may be logical to locate the presets at (L,I) values at or near (L,I) values that were noticed during such preliminary testing to have suitable or good neural potential performance (e.g., low scores) to ensure the further testing of values that seem promising.

A data set 230 is formed in the clinician programmer 70 as the algorithm 200 runs, and includes the electrode configurations necessary to form stimulation at the prescribed longitudinal positions, L. For example, and referring to illustration of lead 21 in FIG. 11, notice that longitudinal position L=1 (step i=1 for the first preset value) corresponds to the location of split ring electrodes E11, E12, and E13. Because step 300 only seeks to determine L and I without imparting any directionality (rotation) to the stimulation (i.e., such that the stimulation field 112 is symmetric around the lead), these electrodes E11, E12, and E13 will share the cathodic current equally (33%*−I), effectively placing cathodic pole 120 longitudinally at L=1. More specifically, because I=2 mA at this step i=1, each of electrodes E11, E12, and E13 will receive −0.67 mA at this step. (Note that the actual current provided at the active electrodes may depend upon the resolution providable by the IPG 10. For example, if the IPG 10 can provide current in 0.1 mA increments, each of the electrodes E11, E12, and E13 may receive 0.7 mA, i.e., 0.67 mA rounded to the nearest tenth).

Longitudinal position L=6 (step i=2 for the second present value) corresponds to the location of ring electrode E2, which will receive 100% of the cathodic current (100%*−I) to place cathode pole 120 longitudinally at this position. More specifically, because I=2 mA at this step i=2, electrode E2 will receive −2.0 mA at this step.

Longitudinal position L=3.5 (step i=3 for the third preset value) is directly between ring electrode E4, and the longitudinal position of split ring electrodes E5, E6, and E7. Therefore, to place the cathode pole 120 (virtually) as this position, the cathodic current is shared equally between E4 (50%*−I) and E5, E6, and E7 as a group (with each receiving 16.7%−I). More specifically, because I=3.5 mA at this step i=3, each of electrode E5, E6, and E7 will receive −0.6 mA at this step (rounded), with E4 receiving 1.7 mA (rounded to give a sum total of 3.5 mA). Although not shown, remember that these electrode configurations as reflected in data set 230 are determinable in the external system software 96 using the electrode configuration algorithm described earlier, which can comprise a portion of optimization algorithm 200.

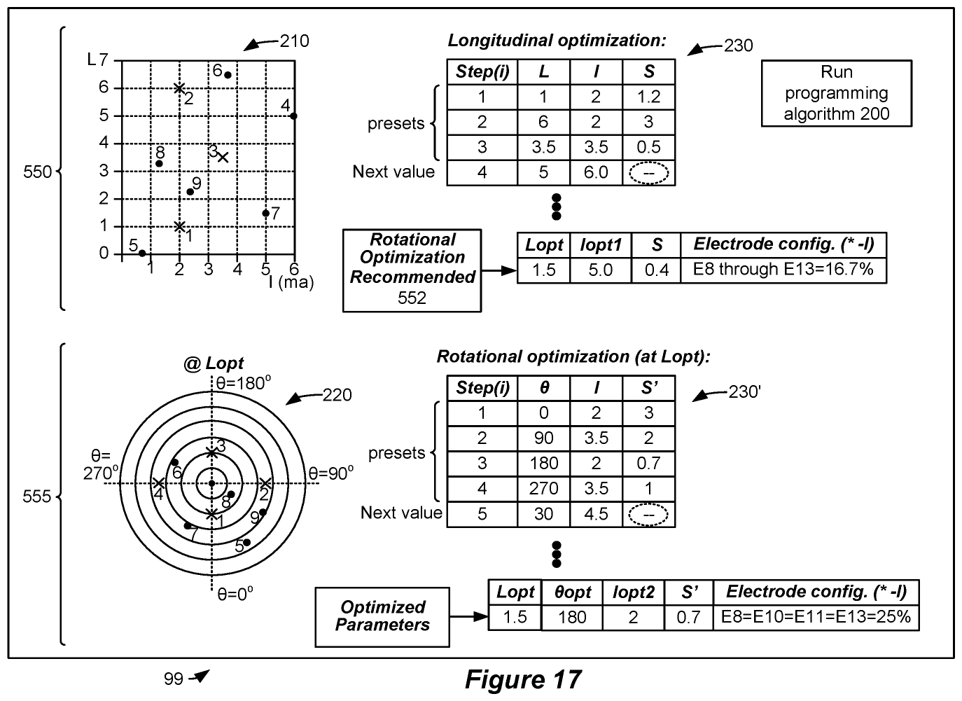

The stimulation parameters as embodied in the (L,I) presets and as determined by the electrode configuration algorithm (the active electrodes; whether they are anodes or cathodes, and the amplitude at each active electrode) are sequentially transmitted to the patient's IPG 10 (along with other non-optimized parameters such as frequency F and pulse width PW) so that the stimulation can be applied to the patient. As each of these stimulation parameters sets are applied, at least one score (S) is then determined for each (315). As noted above, a score can comprise any metric (subjective or objective) that indicates therapeutic effectiveness of and/or the side effects resulting from the stimulation parameters sets. As assumed earlier, a lower score in the depicted example indicates a better result, with 0 being good and 4 poor, although a different scale could be used in which higher numbers are better. The scores (S) once determined for each of the presets are entered into the data set 230 in the clinician programmer 70, such as by having the clinician type the score into the GUI 99 (see FIG. 17). Scores can also automatically be populated into the data set 230 if they are objectively measured and determined. In one example, the score may be indicative of a neural potential (e.g., ERNA) provided at the position of the presets. FIG. 9 earlier described different manners in which neural potentials can be evaluated and if necessary converted into a score S, and as noted earlier, the actual extracted features or their combinations (e.g., peak height) can also be used as a score S. In FIG. 12, notice that the first (L,I) point (1,2) results in a score of 1.2; the second (6,2) results in a score of 3 (poor); and the third (3.5, 3.5) results in a score of 0.5 (good). As discussed later, the scores S can comprise a composite based on a number of metrics that indicate effective and/or side effects, and a plurality of scores can also be determined at each (L,I) value, but this detail is not yet shown for simplicity.

After sequentially applying stimulation according to these presets and determining and recording their scores S after patient testing, the optimization algorithm 200 can determine a best of the (L,I) values (Lopt, Iopt1) based on the scores at those points (320). As explained further below, as the algorithm 200 iterates, more (L,I) values will be tested and scored, and (Lopt, Iopt1) can be updated accordingly at this step. At this point, after only testing the presets, (Lopt, Iopt1) is determined at step 320 to be (3.5,3.5), because this tested value yields the best (e.g., lowest) score (of 0.5).

Next, the algorithm 200 determines whether one or more stopping criteria have been met (325). If a stopping criterium has been met (325), the algorithm 200 may stop iterating—i.e., determining and testing further (L,I) values—and at this point (Lopt, Iopt1) is established. Any number of stopping criteria can be used. For example, the algorithm 200 may decide to stop: if a last determined (L,I) value is too close to other values that have been tested (note that a different distance equation and/or weighting could be used for a stopping determination versus a next-point prediction determination as described further below); if the scores at a number of proceeding values are poor (suggesting that the algorithm is no longer suggesting new (L,I) values to useful effect); if a score at the last selected value is significantly good (suggesting that the algorithm can simply select this last value as the optimal point); if a maximum number of steps (i) has been reached; etc. The stopping criteria need not be automated in the algorithm 200. For example, a stopping criterium may simply comprise the clinician deciding that no further steps are required.

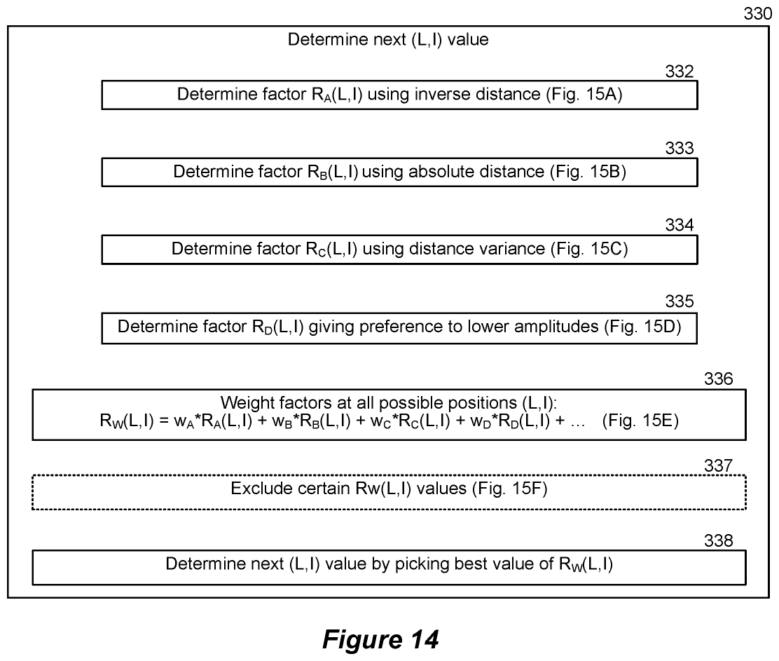

If a stopping criteria has not been met (325), the algorithm 200 proceeds to determine a next (L,I) value to be tested (330) in a next iteration (step i=4). Details involved in choosing this next (L,I) value are shown first with respect to FIG. 14. The algorithm 200 computes and considers one or more factors, and the illustrated example considers four factors R_{A}, R_{B}, R_{C}, R_{D}, although more or fewer factors could be used. Each of these factors is preferably calculated at all possible (L,I) points (332-335) in the L,I parameter space 210, although certain (L,I) values can also be excluded (337), as explained further below. Factors R may be determined based on the distance to all previously-tested (L,I) values, the scores S at those points, or based on other considerations explained further below. When more than one factor is used, the factors can be weighted (which can include normalizing or ranking, as explained further below) and summed (R_{W}; 336) at each (L,I) value, as explained further below. A next (L,I) value to be tested can be determined by picking the weighted factor R_{W}(L,I) having the best (e.g., lowest) value (338), again as explained in further detail below.

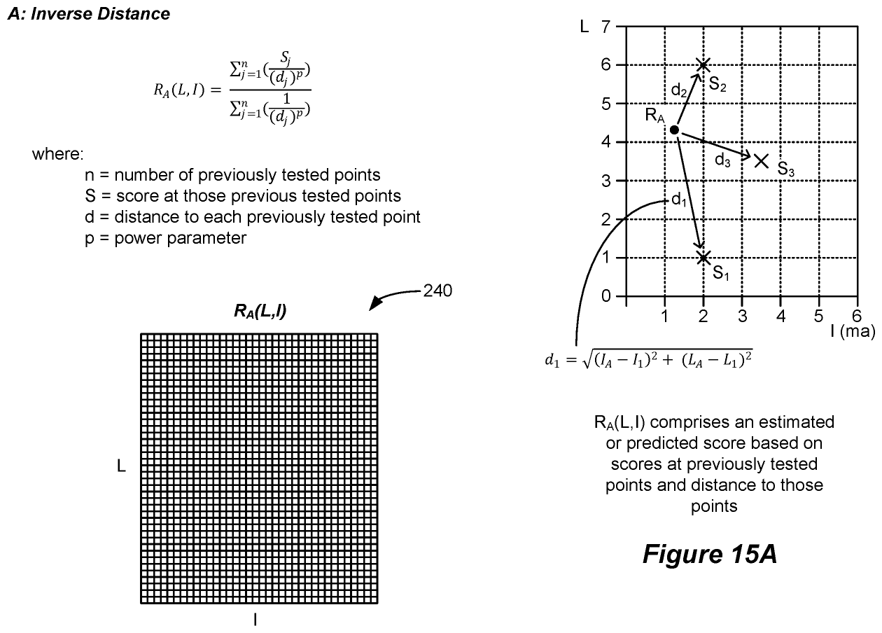

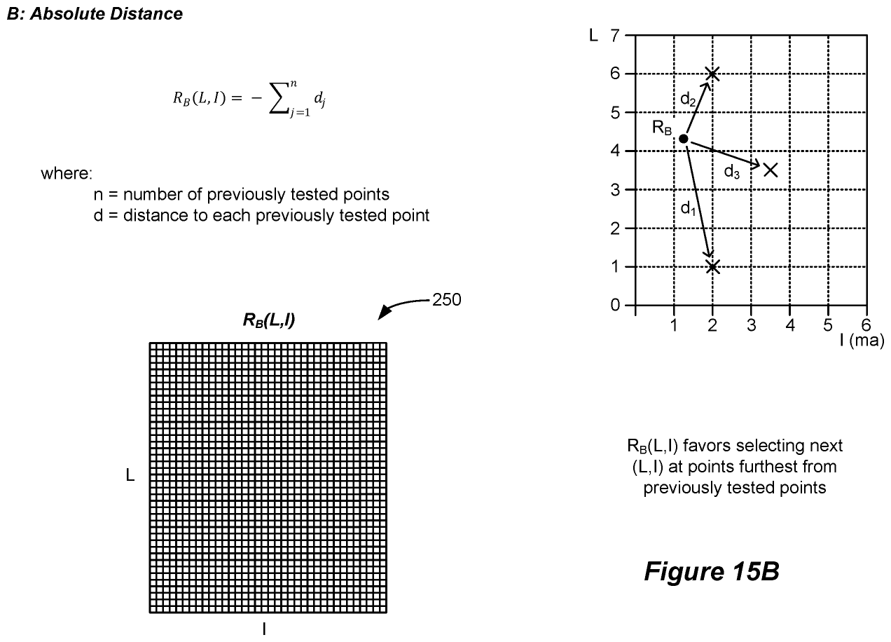

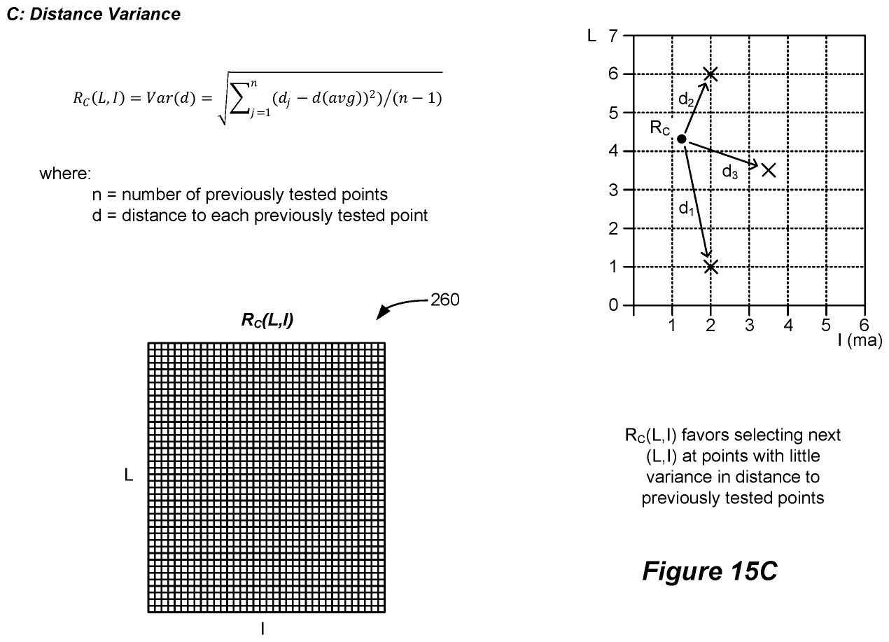

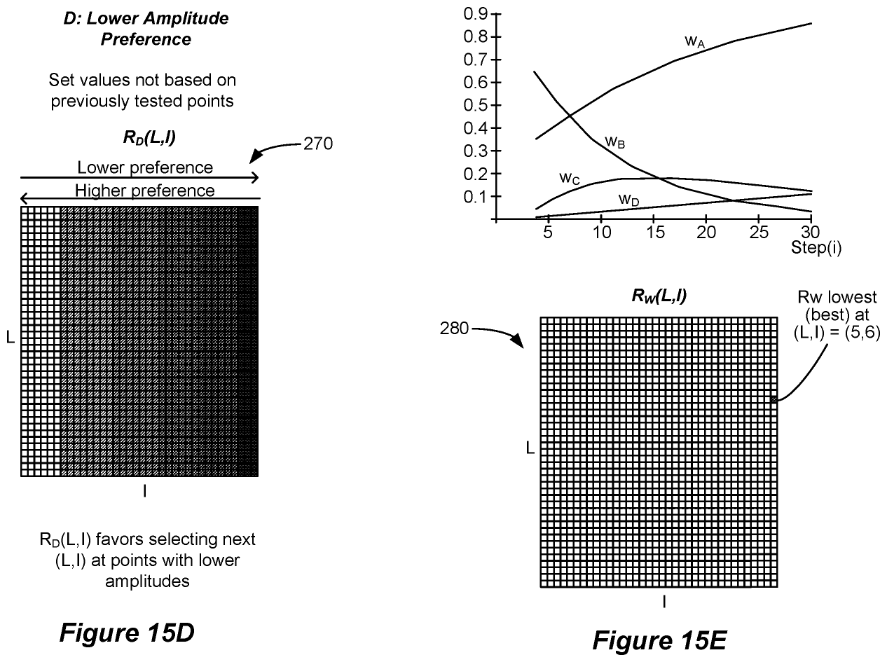

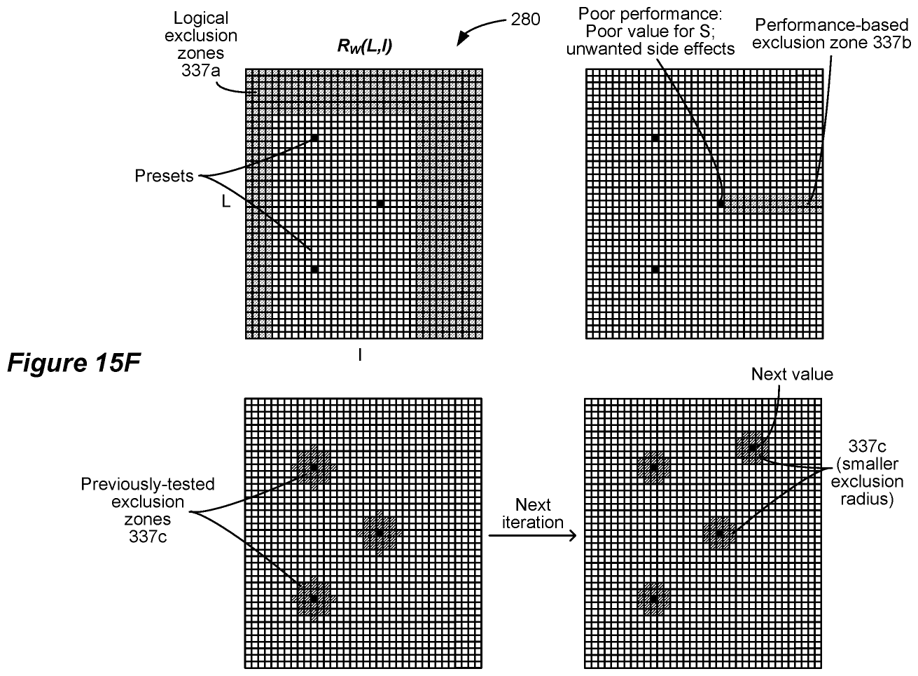

Step 332 calculates factor R_{A} for all (L,I) positions using an inverse distance metric, as shown in FIG. 15A. The calculated value for R_{A} at each (L,I) position in L,I parameter space 210 is represented by data set 240, which is determined and stored in the clinician programmer 70 (or other external system) as the algorithm 200 runs. Note that the number of entries comprising R_{A} data set 240 depends on the resolution at which both I (e.g., 0.1 mA) and L (e.g., in tenths) are defined in the system, as well as the extent to which L,I parameter space 210 is searched. The equation for calculating R_{A} at each (L,I) point in data set 240 is shown in FIG. 15A. Note that factor RA relies on both the previous position of previously-tested (L,I) values (in steps i=1-3) as reflected in the inverse distance 1/dⱼ from each (L,I) point to the previously-tested points. R_{A} also relies on the scores Sᵢ (S₁, S₂, S₃) at each of those previously-tested points, and as noted above such scores can be based on measured neural potentials. In this example, (L,I) values having lower values for R_{A} are more likely to selected as a next (L,I) value to test, as explained further below.

Note as shown in the equation in FIG. 15A that distance d can be determined in a Euclidian fashion, and can comprise the square root of the sum of the differences (in L and I) squared. However, distance could be computed in other fashions. For example, stimulation field modelling (SFM) can be used to model a volume of activation (VOA) in the tissue both at a given (L,I) point and each of the previously-tested points, with a centroid or some other relevant point within those VOA used to determine the distances. SFM modelling may also be beneficial to determining whether stimulation will overlap with an exclusion zone, as discussed further below with respect to FIG. 15F. Variable ‘p’ represents a power parameter that tends to accentuate the distances di and can be empirically set (e.g., to 5 in one example). In effect, factor R_{A} comprises an estimated or predicted score at other (L,I) values that have not yet been tested, and such estimated or predicted scores are based upon the scores at previously-tested positions, as well as the inverse distances to those points. Note that actual calculated values for R_{A} at each (L,I) point are not shown in data set 240. Different prediction-based calculations could be used to determine factor R_{A} as well.

Returning to FIG. 14, step 333 determines a second factor R_{B} for all (L,I) positions using an absolute distance metric, as shown in FIG. 15B. This factor, generally speaking, tends to favor selection of a next (L,I) point that is furthest away from previously-tested (L,I) points, and is thus beneficial in that it encourages the algorithm to select a next (L,I) point for testing at locations in L,I parameter space 210 that haven't yet been tested. The calculated value for R_{B} at each position (L,I) is represented by data set 250, which is determined and stored in the clinician programmer 70 as the algorithm 200 runs. The equation for calculating R_{B} at each (L,I) point in data set 250 is shown in FIG. 15B, and comprises the sum of the distances dⱼ from each (L,I) point to the previously tested points. Because it is desired that lower values for R_{B} are preferred when selecting a next (L,I) value to be tested in the depicted example, this sum is made negative, such that (L,I) values with longer summed distances to previously-tested points are more likely to be selected. However, other means can be used to translate larger summed distances into lower values for R_{B}. Note that R_{B}, unlike R_{A}, does not rely on the scores Sᵢ at previously tested points. Again, actual values for R_{B} at each (L,I) point are not shown in FIG. 15B.

Returning to FIG. 14, step 334 determines a third factor R_{C} for all (L,I) positions using a distance variance metric, as shown in FIG. 15C. This factor, generally speaking, tends to favor selection of a next (L,I) point that is most equidistant from previously tested (L,I) points. The calculated value for R_{C} at each position (L,I) is represented by data set 260, which is determined and stored in the clinician programmer 70 as the algorithm 200 runs. The equation for calculating R_{C} at each (L,I) point in data set 240 is shown in FIG. 15C, and simply comprises the variance of the distances dⱼ from each (L,I) point to the previously tested points, with lower values for R_{C} indicating (L,I) points more likely to be selected as the next (L,I) value to be tested. Note that R_{C}, like R_{B}, does not rely on the scores Sᵢ at previously tested points. Again, actual values for R_{C} at each (L,I) point are not shown in FIG. 15C.

Returning to FIG. 14, step 335 determines a fourth factor R_{D} for all (L,I) positions using a preference for lower amplitudes, as shown in FIG. 15D. This factor, generally speaking, tends to favor selection of a next (L,I) point that has lower values of amplitude, I. This factor is reasonable to consider as it is generally preferred to provide a patient stimulation that is as low in amplitude as possible. The values for R_{D} at each position (L,I) is represented by data set 270. This data set 270 may be preset and not based on the position of or scores at previously tested values. For example, RD may be higher at (L,I) points having higher amplitudes (e.g., R_{D}=4 when I>5 mA) and lower at (L,I) values having lower amplitudes (e.g., R_{D}=0 when I<1 mA), which biases the algorithm 200 towards selection of a next (L,I) value which is at a lower amplitude.

Algorithm 200 doesn't require the use of all of the factors described in FIGS. 15A-15D, and still other factors that aren't shown could be used as well. These factors could also be computed differently.

Returning to FIG. 14, a weighted factor R_{W} for all (L,I) positions is determined using the factors R_{A}, R_{B}, R_{C}, and R_{D} determined earlier (336). In one example, weights w_{A}, w_{B}, w_{c}, and w_{D} can be multiplied by their associated factors to yield R_{W}(L,I)=w_{A}*R_{A}(L,I)+w_{B}*R_{B}(L,I)+w_{C}*R_{C}(L,I)+w_{D}*R_{D}(L,I). The resulting values for R_{W} at each position (L,I) is represented by data set 280 as shown in FIG. 15E.

The weights w applied to the factors can be varied based on user preferences, and FIG. 15E shows example values. FIG. 15E also shows that the weights w can vary in accordance with the step number i—i.e., how many times the algorithm 200 has iterated to determine a next (L,I) value, as explained further below. For example, when initially determining a fourth (L,I) value to test after the three presets (at step i=4), it may be useful to emphasize factor R_{B}—absolute distance (FIG. 15B)—when determining R_{W} because this factor favors choosing a next (L,I) value that is furthest from the previously-tested presets in L,I parameter space 210. During later iterations and after more (L,I) positions have been determined and scored, it may be more useful to emphasize factor R_{A}—inverse distance (FIG. 15A)—because this factor places greater weight on the scores determined at the previously-tested (L,I) values. Thus, in FIG. 15E, weight w_{A} is lower and weight w_{B} is higher at earlier iterations of the algorithm 200, but w_{A} is higher and w_{B} is lower at later iterations. Although not illustrated, weights w can also be dependent on recorded scores, or in accordance with other static or dynamic factors. One of more of the weights w may be associated with factors Ri that depend on the neural potential measurements discussed earlier (e.g., ERNA), and such measurements themselves can comprise a factor. Such a measurement-associated factor can also vary with step number i in FIG. 15E.

Note that weighting of the factors to arrive at R_{W}(L,I) can involve some amount of processing of the individual factors R_{I}(L,I). In this regard, note that each of the individual factors R_{I}(L,I) may be of different magnitudes, depending on how such factors are computed. As such, it may be beneficial to normalize the different factors R_{I}(L,I) so that their magnitudes are generally equated before these factors are weighted by weights w_{I}. Alternatively, the weights w_{I} themselves may be adjusted to accomplish such normalization, so that the individual contributions provided by w_{I}*R_{I}(L,I) leading to R_{W}(L,I) are generally equal in magnitude. In another alternative, each of the (L,I) values for a given factor R_{I} can be ranked, with for example a best (lowest) value (L,I) being given a best (e.g., lowest) ranking (e.g., 1), and a worst (highest) value (L,I) being given a worst (highest) ranking (e.g., L*I). Ranking each (L,I) value for each factor R_{I} before weighting tends to normalize the values of each of the factors, making their weighting by w_{I} more meaningful.

Returning to FIG. 14, certain R_{W}(L,I) values can be excluded from the R_{W}(L,I) data set 280 (337) prior to selecting a next (L,I) value to be tested (338), and this is explained further with reference to FIG. 15F. Specifically, FIG. 15F shows different examples of (L,I) values that can be excluded from data set 280 and the rationale behind their exclusion. Generally speaking, exclusion prevents the algorithm 200 from selecting (L,I) values to be tested that are likely not helpful in determining optimal values Lopt and Iopt1. Exclusion may involve the algorithm 200 applying different exclusion zones that exclude different ranges of (L,I) values. Exclusion zones may be automated via operation of the algorithm 200, or the algorithm 200 may allow the clinician to define exclusions zones. For example, although not shown, the GUI 99 may allow the clinician to define one or more exclusions zones.

For example, the upper left example of the R_{W}(L,I) data set 280 shows different examples of exclusion zones 337a, which comprises zones of (L,I) values that are logical to exclude from testing for one reason or another. For example, it may be known that (L,I) values at very low amplitudes I (e.g., less that 0.8 mA) are unlikely to be therapeutically effective for the patient. Therefore, all (L,I) values at such low amplitudes may be logically excluded, as shown by the grey shading at the left. Other logical exclusion zones 337a can be defined as well. For example, it may be known from earlier testing in the operating room, or otherwise, that stimulation at particular longitudinal values on the lead 21 are not effective in reducing a particular patient's symptoms, or that stimulation at these longitudinal values creates unwanted side effects. Thus, a zone of L values (e.g., from 6 to 7) may be logically excluded, as shown by the grey shading at the top. Similarly, high amplitude values (e.g., >5 mA) may be logically excluded if they have been observed to be ineffective or side-effect inducing, as shown by the grey shading to the right. In another example, logical exclusion zones can be based on objective measurements, such as neural potential measurements (e.g., ERNA). This was discussed earlier (FIG. 12), and with respect to earlier optional step 305. This optional step 305 can also be implemented in the algorithm 200 at step 337 if desired.

Exclusion zones may also be established based on the results of testing at previous (L,I) values. For example, assume as shown in the upper right of FIG. 15F that poor performance was noted at one or more previously tested (L,I) values (shown here as one of the presets). The poor performance may result from a poor (e.g., high) score (S) determined at this point, and/or because unwanted side effects are observed upon testing at this point. In this instance, it may not be useful to consider other (L,I) values at even higher-amplitude currents, as it might be expected that higher amplitudes will make the performance or side effect worse. Thus, the algorithm 200 automatically, or the clinician manually, may define a performance-based exclusion zone 337b. In this example, exclusion zone 337b excludes all (L,I) values with higher amplitudes from the poor performance value. Exclusion zone 337b also for guard band has excluded some higher-amplitude longitudinal positions around this value as well (at slightly different longitudinal positions from the poor-performing point).

Exclusion zones may 337c be also placed around already-tested (L,I) values, as shown at the bottom left of FIG. 15F. This excludes the algorithm 200 from selecting a next (L,I) value that is at or close to a previously-tested (L,I) values. This is shown first in FIG. 15F relative to the three preset values. In this example, the algorithm 200 excludes (L,I) values that are within a certain distance (e.g., radius) of the presets. The reason for doing so is that the presets have already been tested and scored, and it is therefore not useful at this point for the algorithm 200 to potentially recommend a next (L,I) value to be tested that is close to the presets. Instead, to encourage the algorithm 200 to explore values more distant from the presets, such close (L,I) values are excluded.

As explained further below, the algorithm 200 will eventually iterate to select a new (L,I) value to test and score, as shown at the bottom right of FIG. 15F. This can result in the addition of new exclusion zones, or the modification of previously-determined exclusion zones to now re-include (L,I) points that were previously excluded. For example, after a next (L,I) value is determined and scored, there are now four (L,I) points that have been tested (including the original three presets). Notice that the exclusion zones 337c around each of these points have a reduced radius. This can be useful because while the algorithm 200 generally seeks to select distant next (L,I) values as it iterates, it also does not want to ignore potential (L,I) values of interest that were excluded in earlier iterations simply because these values were close to already tested values. As such, and generally speaking, the exclusion zones may be modified by the algorithm 200 as it iterates, and the shape and size of such zones may change based on step number, i.

To summarize, the algorithm 200 can apply various exclusion rules 337 to exclude one or more less-meaningful values to prevent such values from being next selected for testing, thereby increasing the chances that the algorithm 200 will test values that are more meaningful. FIG. 15F shows just some examples of exclusion, but other exclusion rules could be used by the algorithm as well. Note that exclusion of (L,I) points can also occur at different points during the algorithm 200. For example, certain (L,I) values could have been excluded when the data sets 240-270 for the individual factors RA-RD were determined in steps 332-335.

Although not shown in FIGS. 15A-15F, any of the data sets 240-280 can be displayed to the clinician on the GUI 99. In a useful example, the values of the data at each of the (L,I) values can be mapped to a color, thus allowing the data sets 240-280 to appear as “heat maps” whereby data values and general trends can easily be seen in the data. The ability to view heat maps could be added for example to the GUI shown in FIG. 1, discussed later.

Referring to FIG. 14, once Rw is determined at each of the (L,I) values, perhaps with some (L,I) values excluded (305, 337), a best R_{W}(L,I) value is selected (338), which determines the next (L,I) value to be tested (330). The best R_{W}(L,I) value can be determined by the algorithm 200 upon reviewing the non-excluded various values for R_{W} in the data set 280 (FIG. 15E), and selecting the (L,I) value associated with that best (e.g., lowest) value. (Again, and depending on how the factors are processed, a best R_{W}(L,I) value may also have a highest value). In the example of FIG. 15E, the lowest R_{W}(L,I) value is assumed to occur at value (5.0, 6.0), which then comprises the next (L,I) value to be tested (at step i=4). Note that if there is more than one best R_{W} value in data set 280 (e.g., two or more R_{W} values having the same lowest value), the algorithm 200 can be programmed with tie-breaking rules to arrive at a single next (L,I) point. For example, the algorithm 200 may prefer to pick a next (L,I) value that has a lowest amplitude, or that is furthest from all previously tested values, etc.

Note that prior data determined upon testing of the patient can be used in place of, or can comprise, a preset value. Further, presets do not necessarily need to be pre-established at set (L,I) points. Instead, the clinician can simply start testing at a particular (L,I) value, record a score, etc. Eventually, when the algorithm 200 has received enough scores at previously-tested (L,I) values, it can begin to automatically determine next values at step 330, and the algorithm can begin to iterate.

Referring again to FIG. 12, once the next (L,I) value has been determined (330), that value (5.0, 6.0) can be populated into data set 230 as a next step (i=4) and thus as a next point to be applied to the patient (340) and scored by the clinician (345). Again, the electrode configuration algorithm described earlier can determine an electrode configuration suitable to position stimulation at this new longitudinal position. For example, at new (L,I) value (5,6), notice that a ring electrode E3 is located at L=5.0 (FIG. 11), and so that electrode alone will receive all of the cathodic current (100%*−I, or −6.0 mA when I=6.0 is also considered). Once the electrode configuration is determined, stimulation parameters corresponding to this next (L,I) value are transmitted to the IPG 10 and applied to the patient (340) similarly to what was described earlier for the preset (L,I) values. At least one score (e.g., S=1) is then determined for stimulation occurring at this new (L,I) value (345) and input into the data set 230 as described earlier. This score S as before can be based on subjective or objective (e.g., ERNA) measurements.

At this point, the algorithm 200 can return to step 320, where a best of the tested values (Lopt, Iopt1) is determined and/or updated. As described earlier, this involves looking at the scores associated with each of the previously-tested points (in steps i=1 to 4 to this point). The best (e.g., lowest) of these scores (0.5) is associated with step i=3, and so (Lopt, Iopt1) remains (3.5, 3.5), which is not updated.

As the algorithm 200 continues, it again determines if one or more stopping criteria have been met (325). Assuming this doesn't occur, the algorithm 200 determines a next (L,I) value to be tested (330) in a next iteration of the algorithm (step i=5). Determining this next (L,I) value essentially occurs as described earlier by determining factors R_{A}-R_{D} at all points (L,I) (perhaps excepting excluded points). However, notice that there are now more previously-tested points (L,I) to consider (i.e., four, instead of the initial three presets), meaning that the sums in the equations shown in FIGS. 15A-15C involve additional terms (n=4). Again, the factors RA-RD are weighted to determine R_{W}(L,I), and a next (L,I) value is selected (0, 0.8) for testing using a best value for R_{W} (see data set 230, FIG. 12). This next (L,I) value is applied (340) and scored (345) (S=2); (Lopt, Iopt1) is determined and possibly updated (320), etc. The effect of such successive iterations of the algorithm 200 is shown in the data set 230 and the L,I parameter space 210 of FIG. 12, which shows the (L,I) values that were tested at each iteration (i) of the algorithm 200, and how they logically and efficiently traverse the L,I parameter space 210 as dictated by factors RA-RD and their weightings. Note that the data shown in FIG. 12 is fictitious and only provided to help illustrate operation of the algorithm 200.

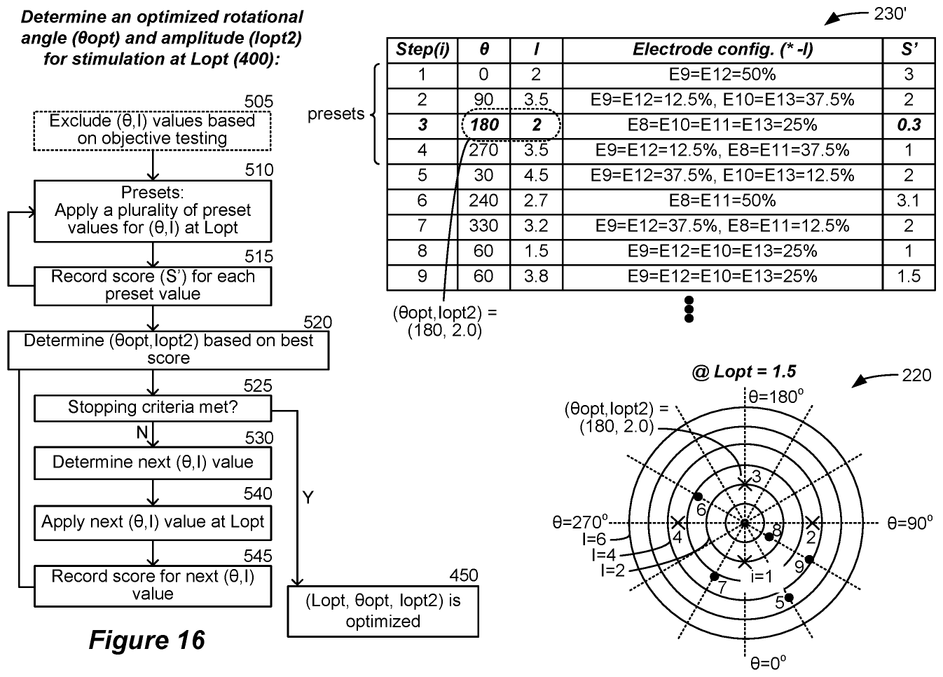

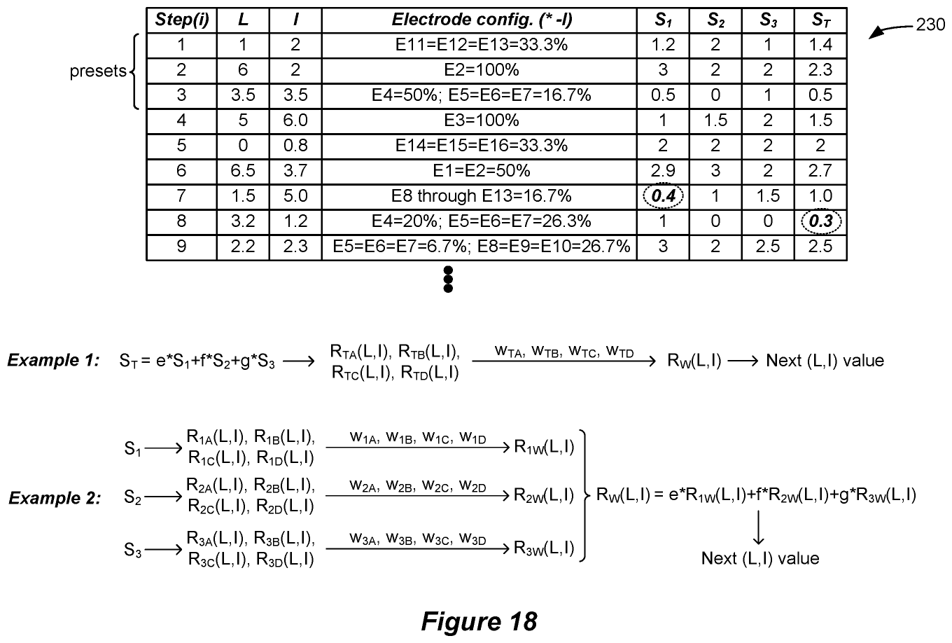

Once a stopping criterium has been met (325), an optimal value of (L,I)—(Lopt, Iopt1)—is determined, which would comprise the (L,I) value determined and updated earlier during step 320. In the illustrated example of the data set 230 in FIG. 12, it is assumed that the algorithm went through nine iterations (step i=9) before reaching a stopping criterium. Review of the scores at each of the tested (L,I) values yields a best (lowest) score of 0.4, corresponding to Lopt=1.5, Iopt1=5.0 mA (step i=7). Note that there may be more than one best value: for example, two (L,I) values may have the same lowest score. In this case, and although not illustrated, the algorithm 200 may employ tie-breaking rules at step 320 to select a single optimal (L,I) value. For example, from amongst the various potential (L,I) values that are tied, the (L,I) value with the lowest amplitude I, or the lowest energy consumption, may be selected. If more than one score is made at each of the tested values, a point discussed further below with respect to FIG. 18, a value with a best average score, or a best single score, may be selected. It may also be preferred or not preferred to select a (L,I) at a particular longitudinal location. For example, it may be preferred to select as optimal an (L,I) value at a longitudinal value that is proximate to split ring electrodes, as this may allow the algorithm 200 to further optimize the rotational angle of the stimulation, as discussed further below with reference to FIG. 16. Conversely, it may be preferred to select as optimal an (L,I) value at a longitudinal value that is not proximate to split ring electrodes; this may simplify optimization because rotational angle does not need to be optimized at such longitudinal positions. Other factors may also be used to break a tie between the scores of (L,I) values reflected in data set 230. For example, the optimal (L,I) may be selected as that having the least side effects, or based on other factors of convenience or efficacy.

Note that (Lopt, Iopt1), while optimized for the patient in the manner explained above, is not necessarily the best (L,I) value for the patient: some other (L,I) value not suggested as a next value by the algorithm 200, and therefore not tested, might actually correspond to a best value (e.g., lowest score S). Nevertheless, (Lopt, Iopt1) can still be said to be optimized for the patient, because the algorithm 200 still searches the L,I parameter space 210 efficiently to arrive at a best value of (Lopt, Iopt1) for the patient. In this sense, (Lopt, Iopt1) can be said to be optimized, or comprise an optimal value, for the patient.

At this point, the optimization algorithm 200 can determine whether the rotational angle θ at which stimulation will be applied (Lopt) should also be optimized. This depends on the determined position of Lopt, and in particular whether Lopt is proximate to split ring electrodes (370), which can require the algorithm 200 to consider the shape and placement of the electrodes on the lead. Referring again to FIG. 11, notice that if Lopt≥4.0, Lopt is not proximate to any split ring (directional) electrodes on lead 21, and there is therefore no reason to optimize rotational angle. In this circumstance, optimization by the algorithm 200 is complete, with (Lopt, Iopt1) determined as optimal for the patient (380). In other words, an optimal stimulation parameter set has now been determined for the patient: as explained above, from Lopt, the algorithm can determine (using the electrode configuration algorithm) the active electrodes, their polarities, and the percentage of Iopt1 that each active electrode should receive, which along with other non-optimized parameters (e.g., F, PW) comprises the optimized stimulation parameter set.

By contrast, if Lopt<4.0, then split ring electrodes are proximate to Lopt in this example. Note that whether Lopt is proximate to split ring electrodes can depend on the electrode configuration used to set Lopt at that longitudinal position, and whether that electrode configuration involves the use of split ring electrodes. For example, and referring to data set 230, it is seen that split ring electrodes E8-E13 are involved in setting (Lopt, Iopt1)=(1.5, 5.0), which allows the algorithm 200 to conclude that Lopt is proximate to split ring electrodes (370), because at least one split ring electrode is active to fix the position of Lopt.

When the algorithm 200 determines that Lopt is proximate to split ring electrodes (370) as in the depicted example, the algorithm 200 can proceed to determine an optimal rotational angle θopt for the application of stimulation at this longitudinal position Lopt (400), as shown in FIG. 16. Similar to the manner in which longitudinal position was simultaneously optimized (Lopt) with amplitude (Iopt1), the rotational angle of stimulation is also preferably simultaneously optimized (θopt) with amplitude (Iopt2). Because changing the rotational angle θ of the stimulation around the lead 21 changes the tissue receiving the stimulation, it is likely that the amplitude optimized at θopt will be different (Iopt2) from that determined when non-directional stimulation was provided (Iopt1).

Optimizing rotational angle θ in algorithm 200 involves trying different angles θ and amplitudes I at Lopt until θopt and Iopt2 are determined. As before, this process is iterative, and involves analogous steps (510-545) as occurred during longitudinal optimization of the stimulation (FIGS. 12-15F), as explained in the above-incorporated '950 Publication. Optimizing rotational angle θ involves the use of θ,I parameter space 220 (FIG. 11) and data set 230′. As before, in optional step 505, certain (θ,I) values may be excluded from further consideration and testing in the algorithm 200 based on objective testing. In a preferred example, such objective testing can comprise neural potential measurements (e.g., ERNA). In this regard, the algorithm 150 discussed earlier (FIG. 8) can be run at step 505, or the results from algorithm 150 queried if it was run earlier. As described earlier, algorithm 150 tests various positions X to see which might be optimal for stimulation for a given patient, and in particular can provide extracted values or scores at those positions. Because positions X may correspond to different longitudinal positions θ, and because testing may occur at one or more known current amplitude I, the positions X correspond to different (θ,I) values in θ,I parameters space 220. If certain (θ,I) values at Lopt were determined to have poor neural potentials (low extracted features values; high scores S), meaning stimulation at that location is unlikely to provided benefit for the patient, those values can be excluded from the θ,I parameters space 220, although such exclusion isn't shown in the Figures that follow.

Next, preset values (θ,I) (510) are chosen to cover a considerable or relevant portion of the possible (θ,I) values not excluded in θ,I parameter space 220. In the example shown, four presets are used with (θ,I) values of (0°, 2 mA), (90°, 3.5 mA), (180°, 2 mA), and (270°, 3.5 mA). Again, these are just example presets, and different values could be chosen, and different numbers of presets used. Note that the current amplitude of the presets can also be selected in light of Iopt1 as determined earlier during longitudinal optimization (FIG. 12). For example, because Iopt1=5.0 mA was determined earlier, current amplitudes equal to or closer to this value may be chosen for the (θ,I) presets. For example, presets of (0°, 4 mA), (90°, 5.5 mA), (180°, 4 mA), and (270°, 5.5 mA) may be used.

Data set 230′ keeps track of these values, and also stores (again with help of the electrode configuration algorithm) the electrode configurations needed to provide the stimulation at these different rotational locations. Data set 230′ may be a continuation of the data set 230 used during longitudinal optimization (FIG. 12), or it may comprise a separate data set. Similar to what occurred during longitudinal optimization, the algorithm will determine a next (θ,I) value to test using scores taken at the four presets. These details are explained in the '950 Publication, and because they are similar to steps summarized earlier during longitudinal optimization, they are only quickly summarized here.

Returning again to FIG. 16, the preset (θ,I) values are applied to the patient at Lopt (510). This causes the clinician programmer 70 to transmit stimulation parameter sets indicative of θ and Lopt (as reflected in the electrode configuration) and amplitude Ito the IPG 10 so that stimulation can be produced at the prescribed angle and longitudinal position. Determination of the electrode configurations is explained further in the '950 Publication. As each of these presets (θ,I) is sequentially applied to the patient (510), at least one score S′ at each of the presets is determined (515) and entered into data set 230′ using GUI 99 (FIG. 17). Such scores as before may be subjective or objective (e.g., ERNA). Again, note in the description of rotational optimization that follows that variables are given a prime symbol (e.g., S′, R′, w′) to differentiate them from variables used earlier (FIG. 12) during longitudinal optimization.

After sequentially applying stimulation according to these presets and determining and recording their scores S′ after patient testing, the programming algorithm 200 can determine a best of the (θ,I) values (θopt, Iopt2) tested to this point based on the scores S′ provided at each of the previously-tested positions (520). As the algorithm 200 iterates, more (θ,I) values will be tested and scored, and (θopt, Iopt2) can be updated accordingly at this step. At this point, after only testing the presets, (θopt, Iopt2) is determined at step 520 to be (180°, 2) (at step i=3), because this tested value yields the best (e.g., lowest) score (S′=0.3).

Next, the algorithm 200 determines whether one or more stopping criteria has been met (525), and stopping criteria can be similar to those described earlier for longitudinal optimization. If not, the algorithm 200 continues to determine a next (θ,I) value to be tested (530) in a next iteration of the algorithm (step i=5). As before, the algorithm 200 computes and considers one or more factors, such as R′_{A}, R′_{B}, R′_{C}, R′_{D}, although again more or fewer factors could be used. Each of these factors is preferably determined at all possible (θ,I) points (532-535) in the θ,I parameter space 220, although certain (θ,I) values can also be excluded, as explained earlier. The factors used during rotational optimization are not shown in the Figures, but are explained in the above-incorporated '950 Publication. As before, a weighted factor data set R′_{W} for all (θ,I) positions is determined using the factors R′_{A}, R′_{B}, R′_{C}, R′_{D}determined earlier, which can involve the use of weights w′_{A}, w′_{B}, w′_{C}, and w′_{D. (}R′_{W}(θ,I)=w′_{A}*R′_{A}(θ,I)+w′_{B}*R′_{B}(θ,I)+w′_{C}*R′_{C}(θ,I)+w′_{D}*R′_{D}(θ,I)). Once R′w is determined at each of the (θ,I) values (perhaps with some values excluded), a best R′_{W}(θ,I) value is selected, which determines the next (θ,I) value to be tested (530). In the example shown in FIG. 16, the best (e.g., lowest) R′_{W}(θ,I) value is assumed to occur at value (30°, 4.5), which then comprises the next (θ,I) value to be tested (during step i=5). As before, if there is potentially more than one best value R′_{W} in data set 580, the algorithm 200 can employ tie-breaking rules.

Referring again to FIG. 16, once the next (θ,I) value has been determined (530), that value (30°, 4.5 mA) can be populated into data set 230′ as a next step (i=5) and thus as a next point to be tested by the clinician. The electrode configuration algorithm can determine an electrode configuration suitable to position stimulation at this new rotational position and at Lopt, as shown in data set 230′. Once the electrode configuration is determined, this next (θ,I) value (the stimulation parameter set) is transmitted and applied to the patient (540) similarly to what was described earlier for the preset (θ,I) values. At least one score (S′=2) is then determined for stimulation occurring at this new (θ,I) value and is recorded into the data set 230′ (545) using the GUI 99 for example (see FIG. 17).

At this point, the algorithm 200 can return to step 520, wherein a best of the tested values (θopt, Iopt2) is determined and/or updated. (θopt, Iopt2) will remain as (180°, 2.0) (step i=3), because this step shows the best (e.g., lowest) score S′ to this point. Assuming a stopping criterium isn't met (525), the algorithm 200 continues iterating and determines a next (θ,I) value to be tested (530) (step i=6), which is applied 540 and for which a score S′ is recorded 545, etc. The effect of such successive iterations of the algorithm 200 is shown in the data set 230′ and the θ,I parameter space 220 of FIG. 16, which shows the (θ,I) values that are determined and how they logically and efficiently traverse the θ,I parameter space 210 as dictated by factors R′_{A}-R′_{D} and their weightings. Again, the data shown in FIG. 16 is fictitious and only provided to help illustrate operation of the algorithm 200.

If a stopping criterium has been met (525), no further (θ,I) values are determined or tested, and an optimal value of (θ,I)—(θopt, Iopt2)—is determined, which would comprise the (θ,I) value determined and updated earlier during step 520. In the illustrated example, (θopt, Iopt2) corresponds to the lowest score S′ (0.3) when θopt=180° and Iopt2=2.0 mA (step i=3). Notice in this example that (θopt, Iopt2) happens to correspond with one of the presets, but this is coincidental and wouldn't necessarily occur.

At this point, stimulation for the patient has been optimized (450), with Lopt optimized during longitudinal searching, and (if necessary, and depending on Lopt's proximity to split ring electrodes) with θopt and Iopt2 optimized during rotational searching at Lopt. To summarize, optimization algorithm 200 has determined an optimized stimulation parameter set (Lopt, θopt, Iopt2)=(1.5, 180°, 2.0 mA) for the patient. Note that Lopt and ηopt, pursuant to the electrode configuration, defines how this amplitude Iopt2=2 mA should be split between the electrodes. As shown in the data set 230′ (step i=3), the current Iopt2 should be split equally between electrodes E8, E10, E11, and E13, with each of these electrodes receiving −0.5 mA, which will place the stimulation at the optimal longitudinal (Lopt=1.5) and rotational (ηopt=180°) positions relative to the lead 21. Again, other stimulation parameters—such as frequency F and pulse width PW are included as part of the optimized stimulation parameter set, which are assumed to have been optimally determined elsewhere. Like amplitude I, these other stimulation parameters could be optimized using the disclosed technique as well. As was the case with longitudinal optimization, the algorithm 200 may apply tie-breaking rules to select an optimal (ηopt, Iopt2) value from between otherwise equally-valued scores S′ at step 520. Again, note that (ηopt, Iopt2), while optimized for the patient in the manner explained above, is not necessarily the best (θ,I) value for the patient: some other (θ,I) value not suggested as a next value by the algorithm 200, and therefore not tested, might actually correspond to a best value (e.g., lowest score S′) for the patient. Nevertheless, (ηopt, Iopt2) can still be said to be optimized for the patient, because the algorithm 200 still searches the θ,I parameter space 220 efficiently to arrive at a best value of (ηopt, Iopt2) for the patient. In this sense, (ηopt, Iopt2) can be said to be optimized, or comprise an optimal value, for the patient.

FIG. 17 shows the GUI 99 that may be used to implement the programming algorithm 200, and displays information that may be useful to the clinician as the algorithm 200 operates. GUI 99 may include a user-selectable option allowing the clinician to “run programming algorithm 200.” Upon selection of this option, a longitudinal optimization section 550 may be displayed, including aspects of data set 230 and parameter space 210 that are useful in understanding how the algorithm 200 is progressing during longitudinal optimization. As noted earlier, data set 230 displays preset and next-determined values (L,I), and allows the clinician to input the score (S) at each. Once a stopping criterium is met, the GUI 99 can display the determined values for Lopt and Iopt1, along with other useful information such as its (best) score S, and perhaps even the electrode configuration and/or stimulation parameters that places the stimulation at that position.

If the algorithm 200 determines based on Lopt that rotational optimization is recommended (see FIG. 12, 370), this fact can be displayed to the clinician as a selectable option (552) to allow such rotational optimization to commence. Otherwise, the GUI 99 can display that rotational optimization is not necessary, and that optimization is (Lopt, Iopt1) is complete (not shown). If rotational optimization is undertaken, the GUI 99 can display a rotational optimization section 555, including aspects of data set 230′ and parameter space 220 that are useful in understanding how the algorithm 200 is progressing during rotational optimization. As before, data set 230′ displays preset and next-determined values (θ,I), and allows the clinician to input the scores (S′) at each. Once a stopping criterium is met, the GUI 99 can display the determined values for Lopt, ηopt, Iopt2, along with other useful information such as its (best) score S′, and perhaps even the electrode configuration and/or stimulation parameters that places the stimulation at that position. Although not shown, the GUI 99 of FIG. 1 may also include a picture of the lead for which stimulation is being optimized, along with the location of the cathode pole 120 that is formed by the optimized stimulation, similar to what was shown in FIG. 5A.

Notice that programming algorithm 200 addresses problems of determining optimal stimulation for DBS patients. As mentioned earlier, in a typical DBS system there are many combinations of I, L, and θ that that can be tested and scored when determining optimal stimulation parameters, and testing all such combinations is burdensome and impractical during a programming session. Use of the programming algorithm 200 efficiently and automatically selects next values to test, and automatically decides when enough values have been tested. As such, much of the guess work in selecting optimal stimulation parameters is removed, and optimal stimulation parameters can be arrived at efficiently and in a reasonable period of time, such as during a typical programming session.

Many modifications can be made to the programming algorithm 200 as described up to this point. Use of the algorithm 200 has been described as particularly useful when used to determine stimulation parameters for a patient having a directional lead (e.g., 19 or 21) with split ring electrodes at least some longitudinal positions on the lead. With such a lead, both longitudinal optimization and rotational optimization can be useful. However, algorithm 200 may also be used in part to provide only longitudinal optimization or only rotational optimization. For example, only longitudinal optimization aspects of the technique (e.g., FIGS. 12-15F) may be used to determine Lopt in a directional lead 21; rotational optimization may not be necessary, or if necessary could occur via other means. Likewise, only rotational optimization aspects of the technique (e.g., FIG. 16) may be used to determine θopt at a particularly longitudinal position along a directional lead, which may be particularly useful if the longitudinal position of the stimulation along the lead (e.g., Lopt) is already known or has been determined by other means. Even if Lopt is not known, the algorithm 200 may still apply only rotational optimization at a given longitudinal position L of the clinician's choosing.

Furthermore, while the algorithm 200 has been described sequentially as comprising longitudinal optimization followed by rotational optimization, the order could be reversed. Still further, longitudinal optimization and rotational optimization can occur more than once. For example, longitudinal optimization may occur to determine Lopt1; followed by rotational optimization to determine θopt1 at Lopt1; followed by further longitudinal optimization to perhaps further optimize Lopt2 at θopt1; followed by further rotational optimization to perhaps further optimize θopt2 at Lopt2; etc.

The algorithm 200 may also be used with non-directional leads (e.g., 18, FIG. 1B) having only circumferential ring electrodes, or with paddle leads. See, e.g., U.S. Pat. No. 10,149,979 (describing paddle leads in a Spinal Cord Stimulation (SCS) system). In these circumstances, only longitudinal optimization aspects of the technique may be necessary.

FIG. 18 shows another modification to programming algorithm 200. In this modification, the clinician can enter a plurality of scores S at each tested stimulation parameter at each iteration of the algorithm. FIG. 18 illustrates this only with respect to longitudinal optimization for simplicity, although this could also be applied to rotational optimization (FIG. 16). As shown, three scores S₁, S₂, and S₃ are determined at each step, although two or more than three could also be considered. For example, S₁ can comprise a bradykinesia score; S₂ a rigidity score; S₃ may comprise a score determined based on objective testing, such as the ERNA measurements described earlier. Again, these are just examples; other subjectively determined or objectively measured patient outcomes can be scored as noted earlier. See U.S. Patent Application Publication 2021/0196956 (discussing determination of a plurality of scores for a given stimulation parameter set in a DBS application).

In Example 1, the scores S₁, S₂, and S₃ are weighted (per weights e, f, and g) to arrive at S_{T}, which is then used to assist in selecting a next value to be tested. Note that such weighting can comprise averaging the scores S₁, S₂, and S₃. Once S_{T} values are determined at previously tested locations, the algorithm 200 can proceed as before to determine a next value to be tested. Thus, and in light of scores S_{T} determined at earlier steps, data sets R_{TA}(L,I), R_{TB}(L,I), R_{TC}(L,I), and R_{TD}(L,I) can be determined (see FIGS. 15A-15D), and these can be weighted to determine a weighted data set R_{W}(L,I) (see FIG. 15E), which can be used to choose the next value ((L,I) in this case) to be tested by determining a best (e.g., lowest) value in R_{W}(L,I).

Example 2 also involves determining a weighted data set R_{W}(L,I) that can be used to select next values to be tested, although this doesn't use S_{T} to do so. Instead, in this example, each of the individual scores S₁, S₂, and S₃ are processed separately to arrive at a weighted data set associated with that score (R_{1W}(L,I), R_{2W}(L,I), and R_{3W}(L,I)), and then these weighted data sets are weighted again (per e, f, and g) to arrive at data set R_{W}. This example is beneficial because the weights used to form the R_{iW}(L,I) data set for each score Sᵢ can be set differently if desired (e.g., w_{iA}, w_{iB}, w_{iC}, w_{iD}). Again, use of multiple scores S′ can also be used during rotational optimization to determine a next (θ,I) value to be tested.

Regardless of how R_{W}(L,I) is determined and used to select next points to be tested, the algorithm 200 can use any of the scores to ultimately select optimal stimulation parameters (in this case, Lopt and Iopt1). For example, once data set 230 is complete and all values have been tested, S_{T} can be assessed to determine Lopt and Iopt1 in this example, which may make sense as S_{T} comprises a general averaging of the individual scores S₁, S₂, and S₃. If S_{T} is used in this manner, the algorithm 200 would determine that Lopt=3.2, and Iopt1=1.2 mA (step i=8), because this step corresponds to the best (lowest) value for S_{T} (0.3) in data set 230. Alternatively, the algorithm 200 could use any of the individual scores in data set 230 to determine optimal parameters. Assume for example that S₁ scores a particularly important symptom such as bradykinesia. The algorithm 200 may thus use this score S₁ to determine the optimal parameters. If S₁ is used in this manner, the algorithm 200 would determine that Lopt=1.5, and Iopt1=5.0 mA (step i=7), because this step corresponds to the best (lowest) value for S₁ (0.4) in data set 230. S_{T} by contrast may be used only to assist in selecting the next values to be tested, or may not be used at all. Still alternatively, algorithm 200 could assess all scores S₁, S₂, S₃, and S_{T}, and use the best (lowest) value of all of these to select the optimal parameters.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method for optimizing stimulation for a patient having a stimulator device, wherein the stimulator device comprises a lead with a plurality of electrodes for providing stimulation, the method providing test stimulation at a plurality of different combinations of a position on the lead and a value of at least one stimulation parameter, the method comprising:
(a) providing test stimulation at initial of the combinations, and measuring a neural potential in response to the test stimulation for each of the initial combinations;
(b) determining at least one score for each of the initial combinations, wherein one of the at least one scores comprises a neural response score based on a feature of the measured neural potential;
(c) determining a next combination using at least all previously determined neural response scores;
(d) repeating the steps prescribed in steps (a)-(c) for the next combination to determine and test further next combinations until a stopping criterium is met; and
(e) using at least the neural response scores to determine optimal therapeutic stimulation for the patient.

2. The method of claim 1, wherein each measured neural potential comprises an Evoked Resonant Neural Response (ERNA).

3. The method of claim 1, wherein the lead is implanted in a brain of the patient.

4. The method of claim 1, wherein the at least one stimulation parameter comprises stimulation amplitude.

5. The method of claim 1, wherein neural potentials are measured in step (a) using at least one of the electrodes.

6. The method of claim 1, wherein the positions vary longitudinally about the lead.

7. The method of claim 1, wherein the positions vary rotationally around the lead.

8. The method of claim 1, wherein determining the optimal therapeutic stimulation in step (e) comprises determining an optimal combination of a position and a value of the at least one stimulation parameter.

9. The method of claim 1, wherein determining the next combination in step (c) comprises determining at least one factor at each possible combination of positions and values of the at least one stimulation parameter, wherein the at least one factor at each possible combination is computed using all previously determined the neural response scores.

10. The method of claim 9, wherein the at least one factor at each possible combination is further computed using a distance between each possible combination and each of the previously tested combinations.

11. The method of claim 9, wherein the next combination is determined in step (c) as the possible combination having the best at least one factor.

12. The method of claim 9, wherein a plurality of factors are determined at each possible combination, and wherein the factors are weighted to determine a weighted factor at each possible combination.

13. The method of claim 12, wherein the next combination is determined in step (c) using the weighted factors.

14. The method of claim 12, wherein the next combination is determined in step (c) as the possible combination having the best weighted factor.

15. The method of claim 1, wherein a second score is additionally determined for each tested combination in step (b).

16. The method of claim 15, wherein the second score is indicative of a patient symptom, a patient response, or a side effect in response to the test stimulation.

17. The method of claim 15, wherein in step (c) the next combination is determined using all previously determined neural response scores and all previously determined second scores.

18. The method of claim 15, wherein in step (e) the optimal combination is determined using the neural response scores and the second scores.

19. A system, comprising:
a stimulator device comprising a lead with a plurality of electrodes for providing stimulation; and
an external device for optimizing stimulation for a patient having the stimulator device, the external device configured to communicate with the stimulator device to provide test stimulation at a plurality of different combinations of a position on the lead and a value of at least one stimulation parameter, wherein the external device is configured to:
(a) cause the stimulator device to provide test stimulation at initial of the combinations, and to measure a neural potential in response to the test stimulation for each of the initial combinations;
(b) determine at least one score for each of the initial combinations, wherein one of the at least one scores comprises a neural response score based on a feature of the measured neural potential;
(c) determine a next combination using at least all previously determined neural response scores;
(d) repeat the steps prescribed in steps (a)-(c) for the next combination to determine and test further next combinations until a stopping criterium is met; and
(e) use at least the neural response scores to determine optimal therapeutic stimulation for the patient.

20. The system of claim 19, wherein each measured neural potential comprises an Evoked Resonant Neural Response (ERNA).
